(19) United States
(12) Patent Application Publication
(10) Pub. No.: US 2025/0025675 A1
(43) Pub. Date: Jan. 23, 2025

(54) THERAPEUTIC PAYLOAD DELIVERY MECHANISM

(71) Applicant: Atmo Biosciences Limited, Box Hill (AU)

(72) Inventors: James JOHN, Box Hill (AU); Malcolm HEBBLEWHITE, Box Hill (AU); Kyle BEREAN, Box Hill (AU); William KENTLER, Box Hill (AU); Mark KIDDER, Box Hill (AU); Spencer Terry WOOD, Box Hill (AU)

(21) Appl. No.: 18/711,339

(22) PCT Filed: Nov. 18, 2022
(86) PCT No.: PCT/AU2022/051389

§ 371 Date: May 17, 2024

(30) Foreign Application Priority Data
Nov. 19, 2021 (AU) 2021903736
Feb. 4, 2022 (AU) 2022900219

(51) Int. Cl. A61M 31/00

(52) U.S. Cl. CPC A61M 31/002; A61M 2205/33; A61M 2205/50; A61M 2205/82

(57) ABSTRACT

Embodiments include an ingestible capsule comprising a housing including a therapeutic payload carrying compartment; a power supply; a release mechanism; and a sensing mechanism, the sensing mechanism being sensitive to an environment external to the housing; the ingestible capsule being configured for passage through a GI tract of a subject mammal, during which passage: the sensing mechanism is configured to output a signal varying according to a GI tract environment, wherein the sensing mechanism comprises one or more sensors from among: a VOC gas sensor; a TCD gas sensor; a reflectometer formed by a transmission antenna of the ingestible capsule connected in series with a directional coupler configured to measure a reflected signal from the transmission antenna; and an accelerometer; the release mechanism is configured to cause a therapeutic payload to be released into the GI tract from the therapeutic payload carrying compartment at a release timing determined according to the output signal.

### TECHNICAL FIELD

This invention relates to an ingestible capsule for delivering therapeutic payload directly to selected location(s) within the gastrointestinal (GI) tract of mammals including humans.

### BACKGROUND

Gas sensor capsules such as that disclosed in EP3497437A1 house gas sensors within an ingestible capsule so that readings may be made from within the gastrointestinal (GI) tract of a mammal, from which readings concentrations of analyte gases may be determined.

A process for determining type and concentration of particular gases in a multi-gas mixture based on readings taken from within the GI tract by gas sensors on-board an ingestible capsule is disclosed in EP3619526A1.

Therapeutic matter such as drugs, pre-biotics, and pro-biotics, are used to treat a plethora of conditions and illnesses. Drugs administered at a medical clinic may be administered in a number of ways, including via intravenous drip, injected directly into the bloodstream, injected subcutaneously, orally, or by rectal suppository. However, drugs issued by a clinic to be administered by a patient at home, due to lack of specialist expertise and equipment, cannot be administered via intravenous drip or directly into the bloodstream. Of the remaining techniques, subcutaneous injection is unpopular due to reluctance on the part of non-medically trained personnel to administer injections. Such reluctance can lead to drugs being administered incorrectly or not administered at all.

Therefore, drugs (or other therapeutic matter) issued by a clinic to be administered at home tend to be for oral consumption, or for suppository. Suppositories may be unpopular due to discomfort and hygiene concerns associated with rectal administration. Furthermore, rectal administration is inappropriate for a number of drug/treatment scenarios. Oral consumption of conventional drug tablets or capsules may in some cases be the optimum form of administration. However, there are scenarios in which the therapeutic payload is required or most effective if it reaches various stages of the GI tract, which may be beyond the stomach, and the therapeutic matter may be degraded by passage through the stomach.

There are also scenarios in which side effects caused by consumption of certain therapeutic matter may be suppressed or avoided if the therapeutic matter can be released to a stage of the GI tract beyond the stomach.

The amount of therapeutic matter to be consumed may be reduced if the therapeutic matter can be delivered directly to a selected location within the GI tract.

Capsules for oral consumption may be configured to dissolve or otherwise degrade within the GI tract at a particular rate in order to deliver therapeutic matter contained therein to the GI tract at the point of degradation where the capsule wall becomes violated. However, such capsules are inconsistent in their rate of degradation, depending on the environmental conditions within the GI tract. Furthermore, rate of movement of matter through the GI tract is unpredictable, so that location at which such capsules deliver their payload to the GI tract is highly probabilistic.

Efficacy of therapeutic matter is in some scenarios improved by local release directly into a particular region of the GI tract rather than via an ingestible or anal suppository that releases therapeutic matter into a non-determined region of the GI tract. For example, efficacy of pre-biotics and pro-biotics is improved by release into the large intestine.

Systemic uptake of therapeutic matter may have unwanted effects (sometimes referred to as side effects). Dependent on the illness or condition being addressed by the therapy, and the therapeutic matter, some side effects may be reduced in severity or avoided by the local release of the therapeutic matter to a particular region of the GI tract and thus reduced systemic uptake.

It is desirable to provide an ingestible capsule that delivers therapeutic matter to a selected location within the GI tract.

### SUMMARY OF INVENTION

Embodiments include an ingestible capsule, comprising: a biocompatible indigestible housing including a therapeutic payload carrying compartment; a power supply; a release mechanism; and a VOC gas sensor, the VOC gas sensor being exposed to gases external to the housing; the ingestible capsule being configured for passage through a gastrointestinal, GI, tract of a subject mammal, during which passage: the VOC gas sensor is configured to output an output signal varying according to concentration of one or more components of a gas mixture to which the VOC gas sensor is exposed; the release mechanism is configured to cause the therapeutic payload to be released into the GI tract from the therapeutic payload carrying compartment at a release timing determined according to the output signal.

Optionally, the release mechanism is configured to identify in real time an ileocecal junction indicator in the output signal, and to determine the release timing according to the identification of the ileocecal junction indicator.

Optionally, the ileocecal junction transition indicator is a characteristic or combination of characteristics of: a reading, a series of readings, a pattern, a geometric feature, and/or a mathematical feature, in a record of the output signal as a function of time; the characteristic or combination of characteristics being predefined as being caused by transition of the ingestible capsule across the ileocecal junction.

Optionally, the release timing is immediate upon identification of the ileocecal junction indicator; or the release timing is a predetermined period following the identification of the ileocecal junction indicator.

Optionally, release timing is some period after ileocecal junction transition of the capsule in order to allow sufficient sensor data to be collected to indicate or confirm presence in the large bowel. Noting that embodiments may be configured to detect the ICJ transition itself (and thus to conclude that capsule is in the large bowel), or may be configured to detect presence in the large bowel directly.

Optionally, the release mechanism comprises a microcontroller and an actuator; the microcontroller being configured to: during an identification phase, on a rolling basis, record a representation of the output signal for a most recent time period of duration t, and to process the recorded representation of the output signal for the most recent time period of duration t to identify presence or absence of the ileocecal junction indicator; the microcontroller being configured, upon identification of the ileocecal junction indicator, to determine the release timing and to cause the release actuator to release the therapeutic payload from the therapeutic payload carrying compartment.

Optionally, the release mechanism comprises a transceiver and an actuator, and the antenna is configured, during an identification phase, to transmit a transmission signal representing the output signal to remote processing apparatus; the transceiver being configured, upon receipt of a notification signal from the remote processing apparatus, to cause, immediately or at a predetermined delay, the therapeutic payload to be released from the therapeutic payload carrying compartment by the release actuator.

Optionally, the VOC gas sensor comprises a sensor side and a heater side, and the output signal comprises only a sensor side output signal component generated by the sensor side in the absence of any power being supplied to the heater side by the power supply.

Optionally, the VOC gas sensor comprises a sensor side and a heater side, and the output signal comprises a sensor side output signal component generated by the sensor side and a heater side output signal component generated by the heater side.

Optionally, the release mechanism is configured to identify an ileocecal junction indicator in a representation of the output signal by identifying a predefined characteristic in the sensor side output signal component contemporaneous with a predefined characteristic in the heater side output signal component.

Optionally, the VOC gas sensor comprises a heating element and is configured to drive the heating element in pulses, and wherein the VOC gas sensor output signal comprises discrete readings each taken at the same point in the phase of a respective pulse of the heating element.

Optionally, the VOC gas sensor output signal is, or is proportional or directly proportional to, a resistance across a sensor side of the VOC gas sensor.

Optionally, the release mechanism is configured to identify an ileocecal junction indicator in a representation of the output signal by defining a maximum for the output signal during a calibration phase, and during a live phase proceeding the calibration phase identifying a predefined characteristic in the output signal, identifying the predefined characteristic comprising: identifying a decrease in the output signal to a level more than a predefined amount or proportion below the defined maximum; identifying a subsequent increase in the output signal; determining that the output signal does not decrease during a time period of a predefined duration immediately following the identified increase, the decrease being a negative gradient across a preset period or number of readings.

Optionally, the therapeutic payload is a drug, is a pre-biotic substance, a faecal transplant, a biologic, and/or is a pro-biotic substance.

Embodiments include a system comprising an ingestible capsule and a remote processing apparatus: the ingestible capsule, comprising: a biocompatible indigestible housing including a therapeutic payload carrying compartment; a power supply; a release mechanism; and a VOC gas sensor, the VOC gas sensor being exposed to gases external to the housing; the ingestible capsule being configured for passage through a gastrointestinal, GI, tract of a subject mammal, during which passage: the VOC gas sensor is configured to output an output signal varying according to concentration of one or more components of a gas mixture to which the VOC gas sensor is exposed; the release mechanism is configured to cause the therapeutic payload to be released into the GI tract from the therapeutic payload carrying compartment at a release timing determined according to the output signal; wherein the release mechanism comprises a capsule transceiver and an actuator, and the capsule transceiver is configured, during an identification phase, to transmit a transmission signal representing the output signal to remote processing apparatus; the capsule transceiver being configured, upon receipt of a notification signal from the remote processing apparatus, to cause, immediately or at a predetermined delay, the therapeutic payload to be released from the therapeutic payload carrying compartment by the release actuator; and the remote processing apparatus comprising a remote processing apparatus transceiver configured to communicate with the capsule transceiver including to receive the transmission signal representing the output signal, and a processor configured to process the output signal to identify in real time an ileocecal junction indicator in the output signal, and to cause the remote processing apparatus transceiver to transmit the notification signal to the capsule transceiver according to the identification of the ileocecal junction indicator.

Embodiments include a method comprising: providing an ingestible capsule according to an embodiment to a subject mammal for ingestion; processing the output signal of the VOC gas sensor to determine the release timing of the therapeutic payload; causing the therapeutic payload to be released into the GI tract of the subject mammal at the determined release timing.

Embodiments provide a deterministic mechanism for releasing therapeutic payload into a particular region of the GI tract. Embodiments actively sense a location of an ingestible capsule within the GI tract and determine a release timing of therapeutic payload according to the sensed location. Therapeutic payload is released locally into a particular region of the GI tract, for example, the colon.

Efficacy of therapeutic payload is improved by virtue of accurate determination of release location rather than relying on probabilistic methods.

Inconvenience, reluctance, pain, and other issues relating to subcutaneous and anal suppository methods of therapeutic payload delivery are overcome.

Side effects associated with release of therapeutic payload into regions of the GI tract other than the particular region are reduced or avoided. For example, side effects associated with release into the stomach may be reduced or avoided by releasing directly into the large colon. Ingestible capsules disclosed herein provide a mechanism with which to reduce toxicological effects of therapeutic payloads such as drugs by delivering the therapeutic payload directly to the site of action, for example to treat irritable bowel syndrome, as opposed to the pathway being via the bloodstream to the liver.

Embodiments leverage an accurate, reliable, and robust mechanism for determining capsule location.

Embodiments include an ingestible capsule, comprising: a housing, being a biocompatible indigestible housing including a therapeutic payload carrying compartment; a power supply; a release mechanism; and a sensing mechanism, the sensing mechanism being sensitive to the environment external to the housing; the ingestible capsule being configured for passage through a gastrointestinal, GI, tract of a subject mammal, during which passage: the sensing mechanism is configured to output an output signal varying according to the GI tract environment external to the housing, wherein the sensing mechanism comprises one or more sensors from among: a VOC gas sensor; a TCD gas sensor; a reflectometer formed by a transmission antenna of the ingestible capsule connected in series with a directional coupler configured to measure a reflected signal from the transmission antenna; and an accelerometer; the release mechanism is configured to cause a therapeutic payload to be released into the GI tract from the therapeutic payload carrying compartment at a release timing determined according to the output signal.

Optionally, the ingestible capsule comprises processor hardware configured to identify in real time one or more ileocecal junction transition indicators in the output signal of the sensing mechanism, and to determine the release timing according to the identification of the ileocecal junction indicator.

Optionally, the sensing mechanism is a direct gas sensing mechanism comprising a VOC gas sensor, the direct gas sensing mechanism being housed within the capsule in a direct gas sensing portion sealed from other components of the ingestible capsule by a gas impermeable membrane and being exposed to a gas mixture in the environment external to the ingestible capsule via a gas permeable membrane in the housing at the location of the direct gas sensing portion, the output signal output by the sensing mechanism comprising VOC concentration readings of the VOC gas sensor.

Optionally, the VOC gas sensor comprises a sensor side and a heater side, and the output signal comprises only a sensor side output signal component generated by the sensor side in the absence of any power being supplied to the heater side by the power supply.

Optionally, the direct gas sensing mechanism further comprises a TCD gas sensor, or wherein the ingestible capsule is configured to make TCD readings via a heater side of the VOC gas sensor, the output signal output by the sensing mechanism further comprising TCD readings of the VOC gas sensor and/or the TCD gas sensor.

Optionally, identifying the ileocecal junction transition indicator comprises identifying an increase in sensor side VOC gas sensor readings with a contemporaneous increase in H2 concentration, the H2 concentration being derived from TCD readings of the TCD gas sensor and/or heater side readings of the VOC gas sensor.

Optionally, identifying the ileocecal junction transition indicator comprises identifying an increase in sensor side VOC gas sensor readings with a contemporaneous increase in CH4 concentration, the CH4 concentration being derived from TCD readings of the TCD gas sensor and/or heater side readings of the VOC gas sensor.

Optionally, the sensing mechanism is a non-contact sensing mechanism housed in a portion of the ingestible capsule sealed from the environment external to the ingestible capsule by the housing, the non-contact sensing mechanism comprising at least one of an accelerometer and a reflectometer, the reflectometer comprising a transmission antenna connected in series with a directional coupler configured to measure a reflected signal from the transmission antenna, the output signal output by the sensing mechanism comprising accelerometer readings and/or reflectometer readings.

Optionally, the non-contact sensing mechanism comprises the reflectometer, and the ingestible capsule further comprises a diode detector and the diode detector forms a part of the reflectometer, the diode detector being configured to receive the reflected signal from the antenna and to measure an amplitude of the reflected signal, the reflectometer readings in the output signal comprising amplitude measurements of the reflected signal.

Optionally, the ingestible capsule further comprises a quadrature demodulator and the quadrature demodulator forms a part of the reflectometer, the quadrature demodulator being configured to receive the reflected signal from the antenna via the directional coupler and to extract phase information of the reflected signal relative to a carrier signal, the reflectometer readings in the output signal comprising the extracted phase information of the reflected signal.

Optionally, the ingestible capsule further comprises an antenna impedance control mechanism comprising a variable capacitor configured to vary impedance of the transmission antenna, and a controller, wherein the reflectometer and the antenna impedance control mechanism form a closed loop or feedback loop, and wherein the controller is configured to receive the measurements of the amplitude of the reflected signal from a diode detector and to execute a control algorithm to use the amplitude measurements to generate an antenna impedance control signal setting a capacitance of the variable capacitor to vary impedance of the antenna to reduce amplitude of the reflected signal, wherein the reflectometer readings in the output signal comprise readings of the antenna impedance control signal.

Optionally, the closed loop or feedback loop further comprises a quadrature demodulator, and wherein phase information is extracted by the quadrature demodulator and output to the controller, and wherein the controller is configured to use the amplitude information and the phase information to generate the antenna impedance control signal.

Optionally, the non-contact sensing mechanism comprises an accelerometer, and wherein the accelerometer measures an orientation of the ingestible capsule relative to a frame of reference in fixed relation to a gravitational vector, and wherein the microcontroller processes the accelerometer measurements by: determining whether the orientation of the ingestible capsule given by the respective accelerometer measurement is more than a threshold angular displacement from the reference orientation, and if the threshold angular displacement is not met, progressing to the next accelerometer measurement without changing the reference orientation, and if the threshold angular displacement is met, changing the reference orientation to align with the orientation of the ingestible capsule given by the respective accelerometer measurement; wherein identifying an ileocecal junction transition indicator and/or a gastric duodenal transition indicator comprises detecting that a rate of change of the reference orientation meets a detection criterion.

Optionally, the non-contact sensing mechanism comprises an accelerometer, and wherein the accelerometer measures an orientation of the ingestible capsule relative to a frame of reference in fixed relation to a gravitational vector, and wherein the microcontroller processes the accelerometer measurements by: for each of three orthogonal axes in fixed spatial relation to the ingestible capsule derivable from the reading of the orientation, repetitively in respect of each successive accelerometer measurement chronologically: calculating, as a scalar value, a change in the orthogonal axis relative to the gravitational vector from the preceding accelerometer measurement; applying a low pass filter to the calculated changes; recording the cumulative filtered calculated changes; wherein identifying an ileocecal junction transition indicator and/or a gastric duodenal transition indicator comprises detecting a step change meeting a detection criterion in the rate of increase of the cumulative filtered calculated changes.

Optionally, the output signal output by the sensing mechanism comprises accelerometer readings and reflectometer readings, and determining the release timing comprises identifying that an ileocecal junction transition indicator is present in readings from the reflectometer and the accelerometer, including: processing the reflectometer readings and the accelerometer readings to identify the presence of a first ileocecal junction transition indicator in either one of the reflectometer readings and the accelerometer readings, processing the other one of the reflectometer readings and the accelerometer readings to identify a second ileocecal junction transition indicator within a predefined time window of a timing of the first ileocecal junction transition indicator, and in response to identifying the first ileocecal junction transition indicator and the second ileocecal junction transition indicator within the predefined time window, determining the release timing, being either immediate or after a predefined delay.

Optionally, the or each ileocecal junction transition indicator and/or gastric-duodenal transition indicator is a characteristic or combination of characteristics of: a reading, a series of readings, a pattern, a geometric feature, a statistical feature, and/or a mathematical feature, in a record of the output signal as a function of time; the characteristic or combination of characteristics being predefined as being caused by transition of the ingestible capsule across the ileocecal junction or from the stomach into the duodenum.

Optionally, the ingestible capsule comprises a microcontroller, and the release mechanism comprises the microcontroller and a release actuator; the microcontroller being configured to: during an identification phase, on a rolling basis, record a representation of the output signal for a most recent time period of duration t, and to process the recorded representation of the output signal for the most recent time period of duration t to identify presence of the one or more ileocecal junction transition indicators; the microcontroller being configured, upon identification of the presence of the one or more ileocecal junction transition indicators, to determine the release timing and based on the determined release timing to cause the release actuator to release the therapeutic payload from the therapeutic payload carrying compartment.

Optionally, the ingestible capsule comprises a microcontroller, and the release mechanism comprises the microcontroller and a release actuator; the microcontroller being configured to: during an identification phase, on a rolling basis, record a representation of the output signal for a most recent time period of duration t, and to process the recorded representation of the output signal for the most recent time period of duration t to identify presence of the one or more gastric-duodenal transition indicators; the microcontroller being configured, upon identification of the presence of the one or more gastric-duodenal transition indicators, to determine the release timing and based on the determined release timing to cause the release actuator to release the therapeutic payload from the therapeutic payload carrying compartment.

Optionally, the ingestible capsule comprises a microcontroller, and the release mechanism comprises the microcontroller and a release actuator; the microcontroller being configured to: during an identification phase, on a rolling basis, record a representation of the output signal for a most recent time period of duration t, and to process the recorded representation of the output signal for the most recent time period of duration t to identify: presence of the one or more gastric-duodenal transition indicators; and following the identification of the presence of the one or more gastric-duodenal transition indicators, to identify presence of the one or more ileocecal junction transition indicators; the microcontroller being configured, upon identification of the presence of the one or more ileocecal junction transition indicators, to determine the release timing and based on the determined release timing to cause the release actuator to release the therapeutic payload from the therapeutic payload carrying compartment.

Optionally, the therapeutic payload is one or more from among: a drug, a pharmaceutical formulation, a pre-biotic substance, a faecal transplant, and/or a pro-biotic substance.

Optionally, the ingestible capsule comprises a wireless transceiver, and the release mechanism comprises the wireless transceiver and a release actuator, and the antenna is configured, during an identification phase, to transmit a transmission signal representing the output signal to remote processing apparatus; the transceiver being configured, upon receipt of a notification signal from the remote processing apparatus, to cause, immediately or at a predetermined delay, the therapeutic payload to be released from the therapeutic payload carrying compartment by the release actuator.

Optionally, the release mechanism comprises a microcontroller and a release actuator, and the therapeutic payload carrying compartment comprises a section of the ingestible capsule housing and a sealed chamber, an elastic material membrane defining at least a portion of a wall of the sealed chamber, within which sealed chamber the therapeutic payload is sealed; the release actuator comprising an elastic material membrane rupturing mechanism configured, at the determined release timing, to rupture the elastic material membrane thereby unsealing the sealed chamber, and allowing the therapeutic payload to exit the ingestible capsule via one or more apertures in the section of the ingestible capsule housing.

Optionally, the elastic material membrane rupturing mechanism comprises a power source and a heating element, the heating element arranged at least partially within, or against an interior surface of, the therapeutic payload carrying compartment, the at least a portion of the wall of the sealed chamber defined by the elastic material membrane being arranged to contact the heating element; the power source being configured, at the determined release timing and under control of the microcontroller, to transfer energy to the heating element, to increase a temperature of the heating element and by which temperature increase to rupture the elastic material membrane, thereby unsealing the sealed chamber.

Optionally, the power source of the elastic material membrane rupturing mechanism is a supercapacitor configured to be trickle charged by the ingestible capsule power supply over a period of time beginning with an initiation event of the ingestible capsule, and to be caused to release the charge to the heating element at the determined release timing under the control of the microcontroller.

Optionally, supercapacitor and the heating element are impedance matched, or are impedance matched to within a defined tolerance.

Optionally, the heating element is a resistive heater element comprising one or more from among: SMT resistor; metallic resistive wire; nichrome; MEMS heater element.

Optionally, the elastic material membrane rupturing mechanism comprises a power source and a LASER diode focussed on the elastic material membrane, wherein a microcontroller of the ingestible capsule is configured at the determined release timing to activate the LASER diode to rupture the elastic material membrane, thereby unsealing the sealed chamber.

Optionally, the elastic material membrane rupturing mechanism comprises a pre-sprung mechanical needle, wherein a microcontroller of the ingestible capsule is configured, at the determined release timing, to release the pre-sprung mechanical needle causing the pre-sprung mechanical needle to spring into the elastic material membrane causing the elastic material membrane to rupture, thereby unsealing the sealed chamber.

Optionally, the release mechanism comprises a microcontroller and a release actuator, and the therapeutic payload carrying compartment comprises a section of the ingestible capsule housing and a sealed chamber, an elastic material membrane defining at least a portion of a wall of the sealed chamber, within which sealed chamber the therapeutic payload is sealed; the chamber is sealed by a releasable valve; the release actuator comprises a releasable valve releasing mechanism configured, at the determined release timing, to release the releasable valve thereby unsealing the sealed chamber and allowing the therapeutic payload to exit the ingestible capsule via one or more apertures in the section of the ingestible capsule housing.

Optionally, the releasable valve is a kinked hose, wherein the releasable valve releasing mechanism is a shape memory alloy wire or micro motor configured, under control of a microcontroller of the ingestible capsule, to unkink the hose thereby unsealing the chamber and allowing content of the chamber to exit the ingestible capsule via one or more apertures in the housing of the ingestible capsule.

Optionally, the elastic material membrane rupturing mechanism comprises a power source, a shape memory alloy wire, and a rupturing member, the power source being configured, at the determined release timing and under control of the microcontroller, to transfer energy to the shape memory alloy wire, to initiate a phase change at material level of the shape memory alloy wire and thereby to exert a force on the rupturing member to cause the rupturing member to come into contact with, and to rupture, the elastic material membrane, thereby unsealing the sealed chamber.

Optionally, the elastic material membrane rupturing mechanism comprises a motor and a rupturing member, the microcontroller being configured, at the determined release timing, to power on the motor and thereby to exert a force on the rupturing member to cause the rupturing member to come into contact with, and to rupture, the elastic material membrane, thereby unsealing the sealed chamber.

Optionally, the release mechanism comprises a microcontroller and a release actuator, and the therapeutic payload carrying compartment comprises a section of the ingestible capsule housing and a sealed chamber, an elastic material membrane defining at least a portion of a wall of the sealed chamber, within which sealed chamber a liquid diluent is sealed, the therapeutic payload being a lyophilized drug or other therapeutic matter in powdered, dehydrated, or other solid form, and being contained within the therapeutic matter carrying compartment in a space external to the sealed chamber and at least partially defined by the elastic material membrane, the section of the ingestible capsule housing comprising one or more apertures enabling fluid communication between the therapeutic payload carrying compartment and the exterior of the capsule, the one or more apertures being blocked by the elastic material membrane and unblocked following rupture of the elastic material membrane by an elastic material membrane rupturing mechanism at the determined release timing, the rupturing of the elastic material membrane allowing the liquid diluent to mix with the therapeutic payload within the therapeutic payload carrying compartment and to mix with fluids from the environment external to the capsule via the one or more apertures.

Optionally, the elastic material membrane is in a stretched state over a rigid open frame, the rigid open frame defining one or more apertures, the one or more apertures being sealed by the elastic material membrane while the sealed chamber is sealed, and being at least partially open following the rupturing of the elastic material membrane by the release actuator.

Optionally, the rigid open frame defines, in addition to the one or more apertures, one or more further apertures, the one or more further apertures being sealed by an outer cover which is separate from, or a portion of, the housing of the ingestible capsule.

Optionally, the elastic material membrane is a balloon filled with the therapeutic matter or a liquid diluent, and being in an expanded state within the therapeutic matter carrying compartment and covering the one or more apertures in the section of the housing of the ingestible capsule.

Optionally, the ingestible capsule includes an environmental sensor, and the readings include readings of the environmental sensor, the environmental sensor being an environmental temperature sensor, an environmental relative humidity sensor, or an environmental temperature sensor and an environmental humidity sensor; the processing the recorded readings including determining an excretion event timing by detecting an excretion indicator, the excretion indicator being a change in the environmental sensor readings between an internal environmental condition of the subject mammal and an external environmental condition at a location of the subject mammal, the excretion event timing being a timing of excretion of the ingestible capsule by the subject mammal.

Optionally, the ingestible capsule comprising a wireless transceiver configured to transmit data transmission payload away from the ingestible capsule via Bluetooth, Bluetooth Long Range, and/or 433 MHz radio transmission technique, the data transmission payload comprising one or more from among: a record of release timing; a record of excretion timing; a record of the one or more identified ileocecal junction transition indicators; a record of the one or more identified gastric-duodenal transition indicators; a record of an electrode signal indicating rupturing of an elastic material membrane unsealing a chamber containing therapeutic payload or liquid diluent; output signal output by the sensing mechanism; and a metric or metrics representing the output signal output by the sensing mechanism.

Optionally, transmission of the data transmission payload by the wireless transceiver is triggered by one or more from among: determining that an excretion event has occurred; determining release timing; determining release timing and that a predefined delay after release timing has expired; receipt at a microcontroller of the ingestible capsule of an electrode signal indicating rupturing of an elastic material membrane unsealing a chamber containing therapeutic payload or liquid diluent.

Optionally, the sensing mechanism comprises a direct gas sensing mechanism and a non-contact sensing mechanism.

Embodiments include an ingestible capsule, comprising: a biocompatible indigestible housing including a therapeutic payload carrying compartment; a power supply; a release mechanism; and a sensing mechanism, the sensing mechanism being sensitive to an environment external to the housing; the ingestible capsule being configured for passage through a gastrointestinal, GI, tract of a subject mammal, during which passage: the sensing mechanism is configured to output an output signal varying according to the GI tract environment external to the housing; the release mechanism is configured to cause a therapeutic payload to be released into the GI tract from the therapeutic payload carrying compartment at a release timing determined according to the output signal.

The sensing mechanism is sensitive to an environment external to the housing, noting that the sensing mechanism is located within the housing and so the interaction between external environment and sensing mechanism occurs within the housing. Nonetheless, the sensing mechanism is sensitive to changes in the environment in which the capsule is located. In particular, the sensing mechanism is sensitive to composition of the environment in which the capsule is located and/or to interactions between the capsule and the environment in which the GI tract is located. Composition of the GI tract is a combination of fluidic composition including at least one of liquids and gases resident in the GI tract at the location of the capsule, and may also include surrounding tissue. The precise composition will vary as the capsule proceeds along the GI tract, and the extent to which different components of the composition influence the sensing mechanism will vary according to the identity of the components and their arrangement relative to the capsule (and to the antenna position within the capsule) and relative to other components of the GI tract. For example, antenna reflectance is influenced by interfaces between different transmission environments. In the example of an accelerometer, it can be appreciated that the sensing mechanism (including the accelerometer) is sensitive to changes in motion of the capsule caused by physical interactions between the capsule and the environment in which the capsule is located.

Embodiments include an ingestible capsule comprising a housing including a therapeutic payload carrying compartment; a power supply; a release mechanism; and a sensing mechanism, the sensing mechanism being sensitive to an environment external to the housing; the ingestible capsule being configured for passage through a GI tract of a subject mammal, during which passage: the sensing mechanism is configured to output a signal varying according to a GI tract environment, wherein the sensing mechanism comprises one or more sensors from among: a VOC gas sensor; a TCD gas sensor; a reflectometer formed by a transmission antenna of the ingestible capsule connected in series with a directional coupler configured to measure a reflected signal from the transmission antenna; and an accelerometer; the release mechanism is configured to cause a therapeutic payload to be released into the GI tract from the therapeutic payload carrying compartment at a release timing determined according to the output signal.

### DETAILED DESCRIPTION

Embodiments are set out below, by way of example, with reference to the accompanying drawings, in which:

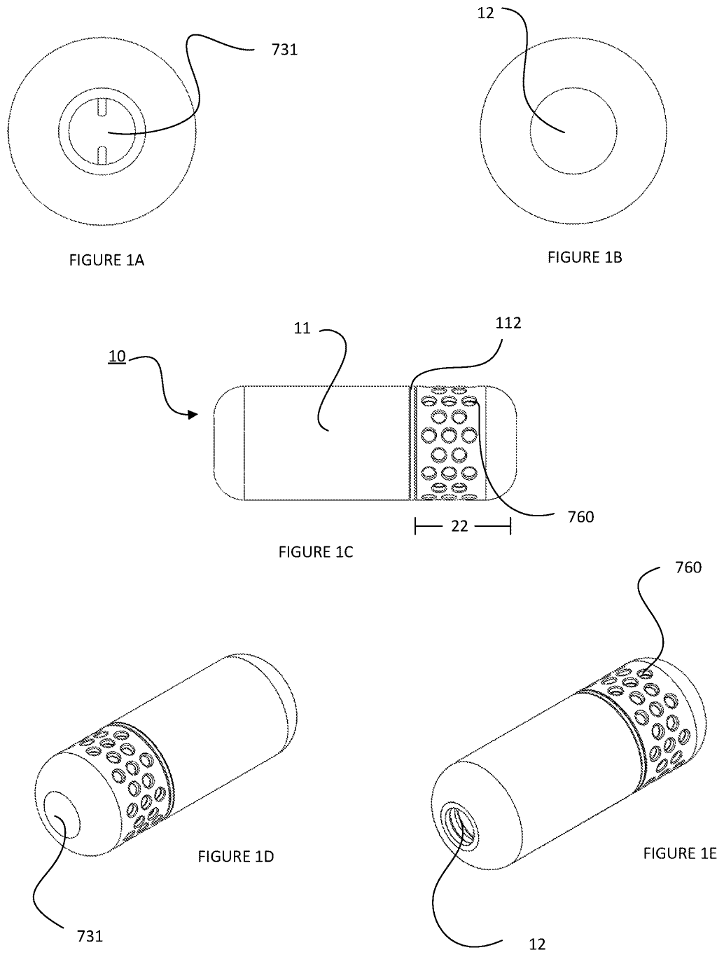

FIGS. 1A to 1E illustrate an exterior of an ingestible capsule of an embodiment;

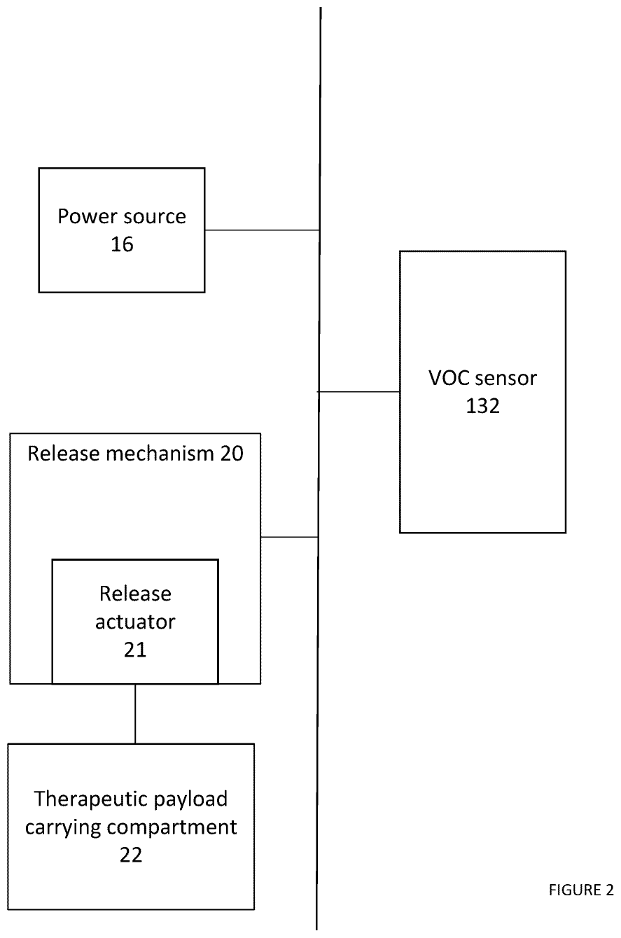

FIG. 2 is a schematic illustration of electronic components inside an ingestible capsule of an embodiment;

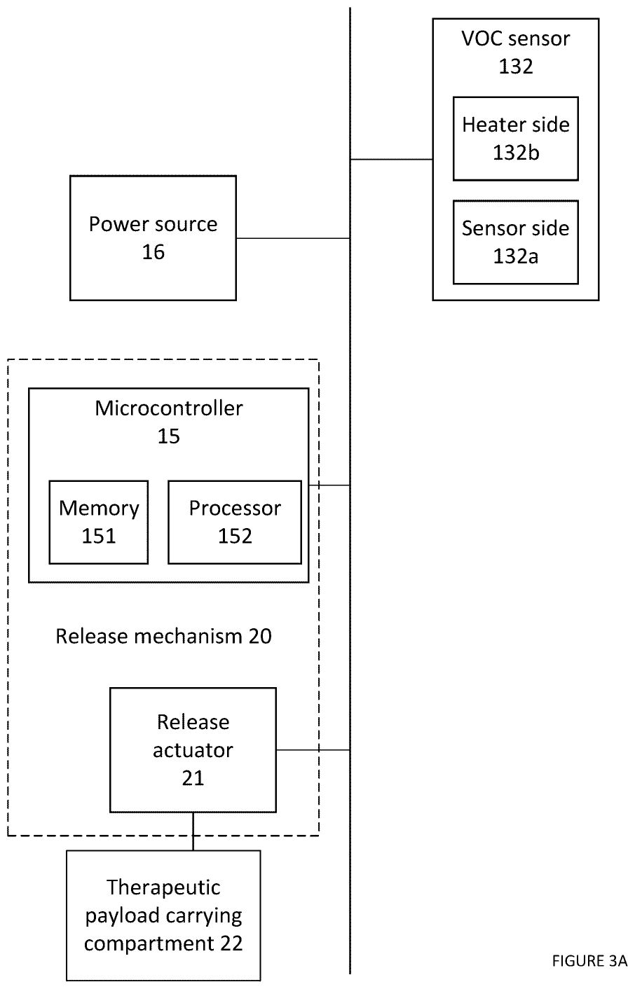

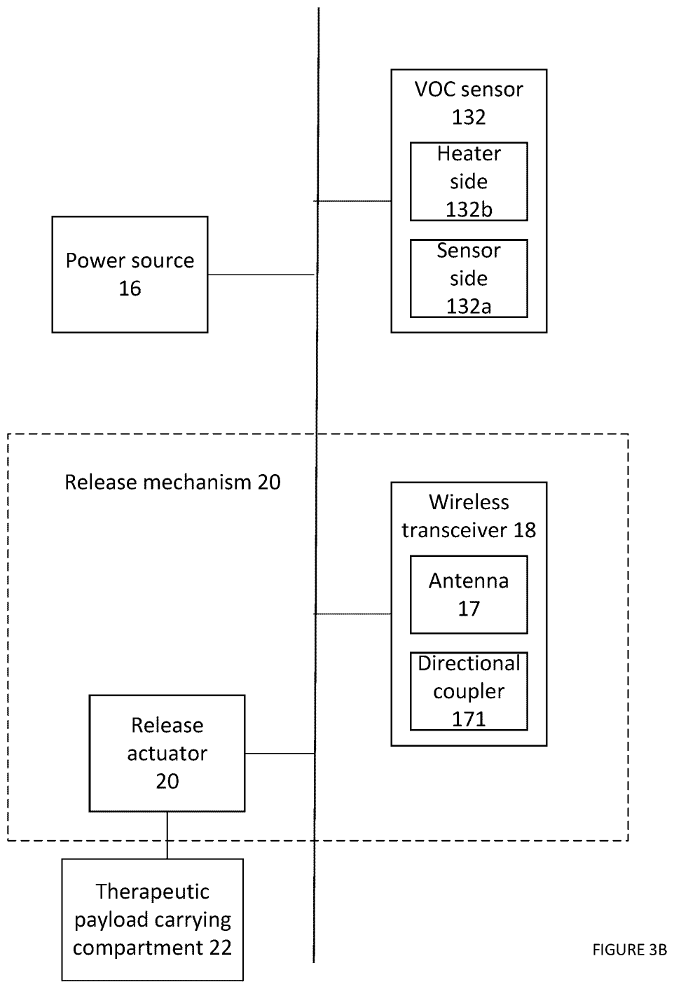

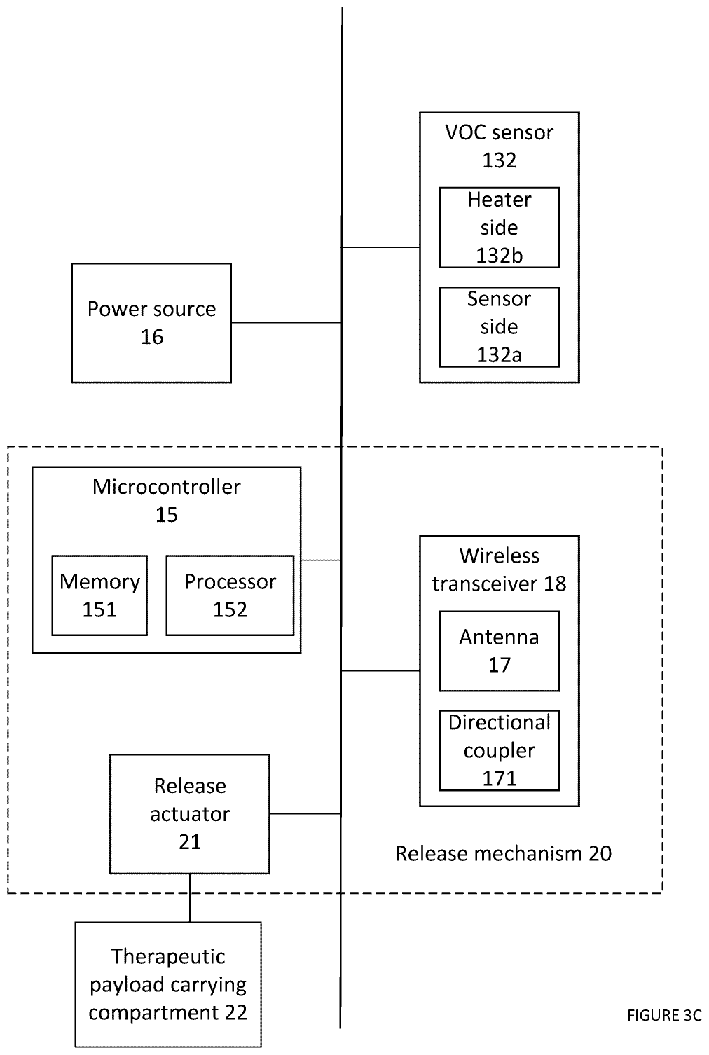

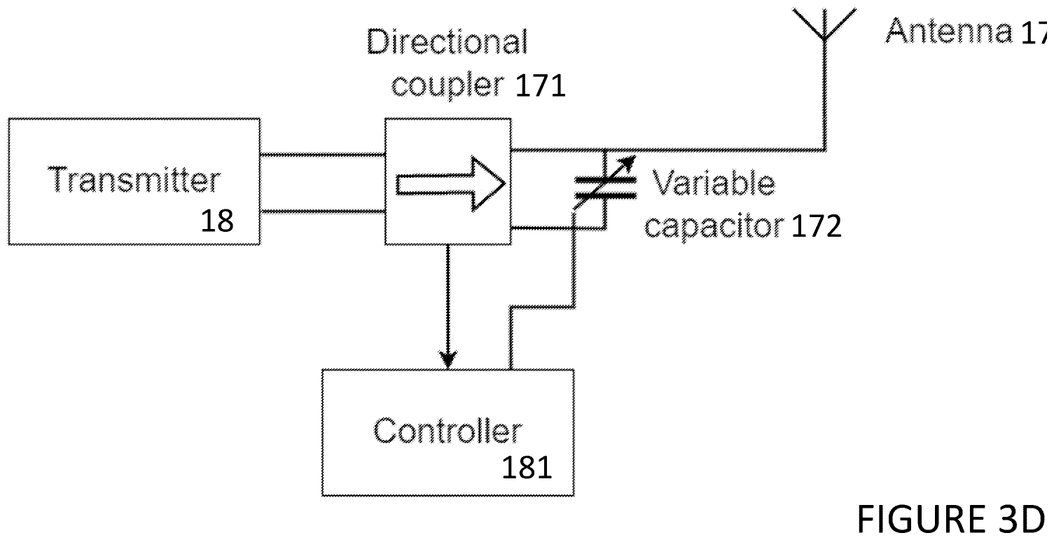

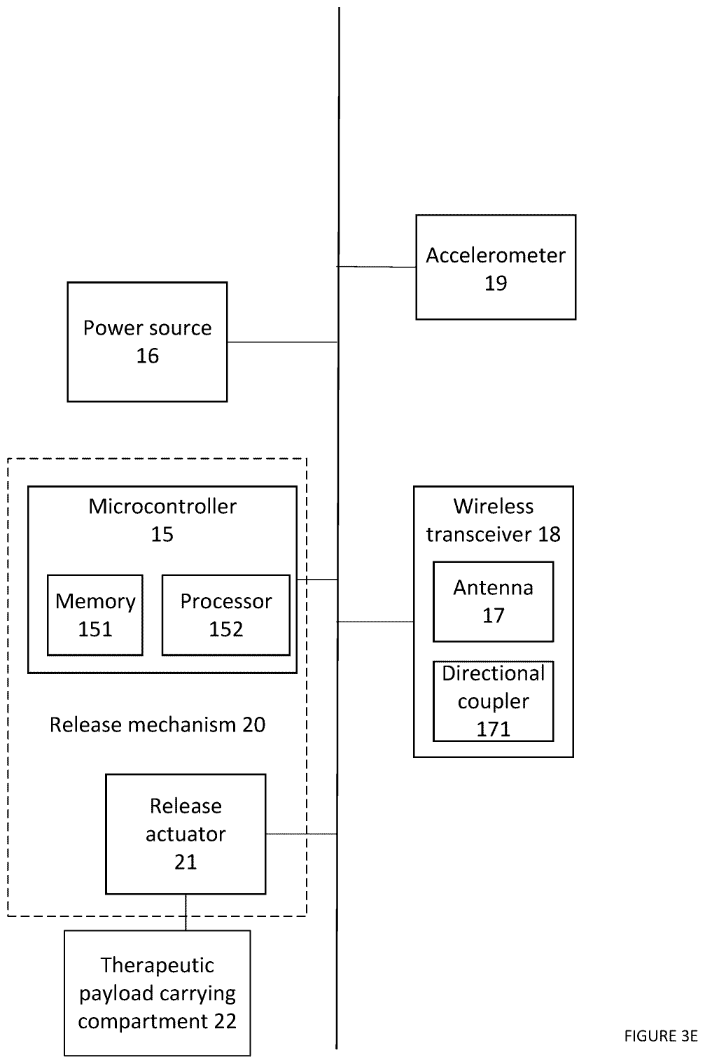

FIG. 3A is a schematic illustration of electronic components inside an ingestible capsule in a local processing embodiment;

FIG. 3B is a schematic illustration of electronic components inside an ingestible capsule in a remote processing embodiment;

FIG. 3C is a schematic illustration of electronic components inside an ingestible capsule in a combined local and remote processing embodiment;

FIG. 3D illustrates a reflectometer;

FIG. 3E is a schematic illustration of electronic components inside an ingestible capsule;

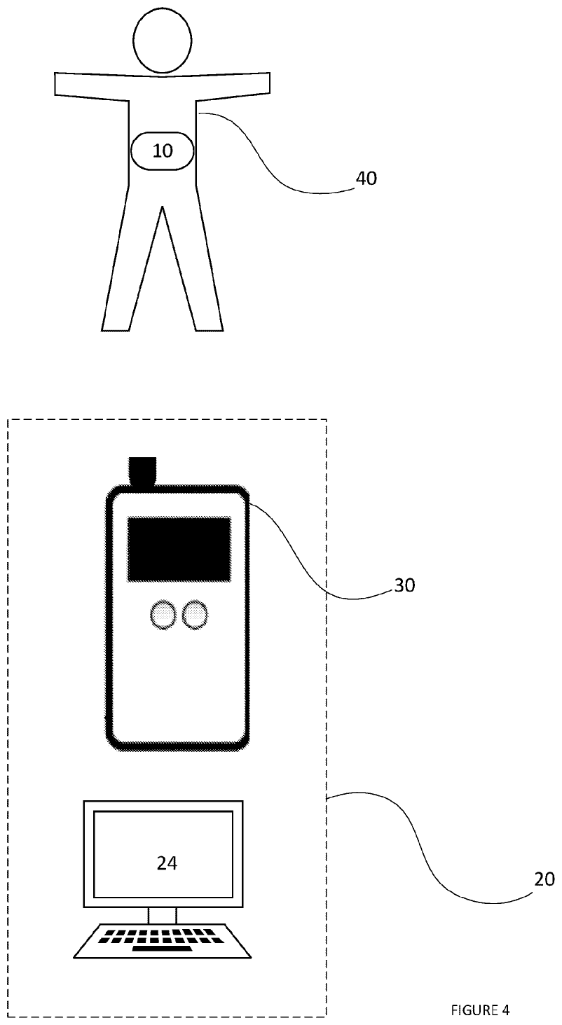

FIG. 4 illustrates a system of an embodiment;

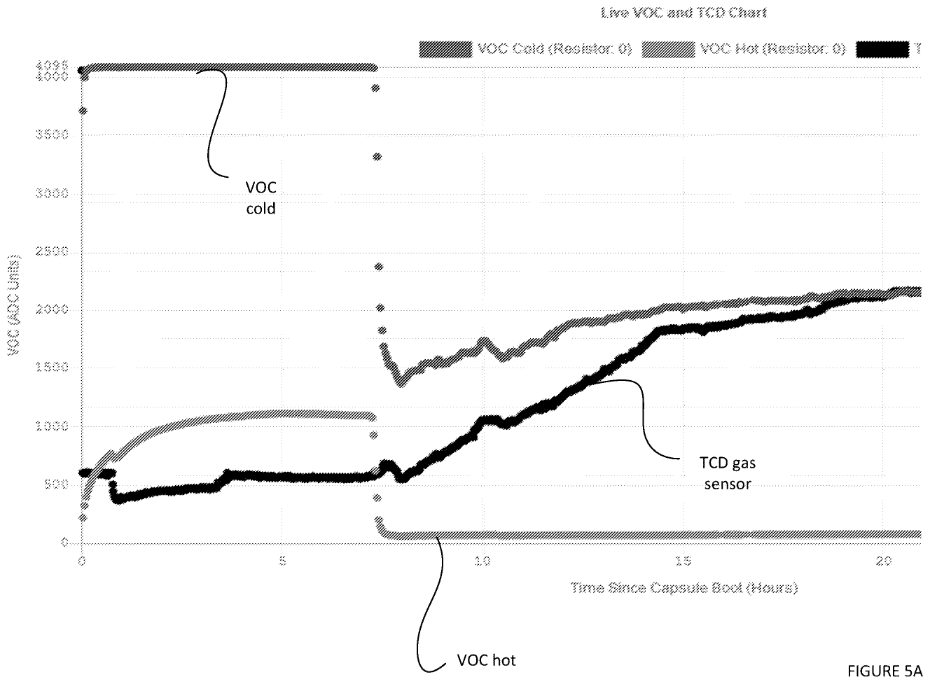

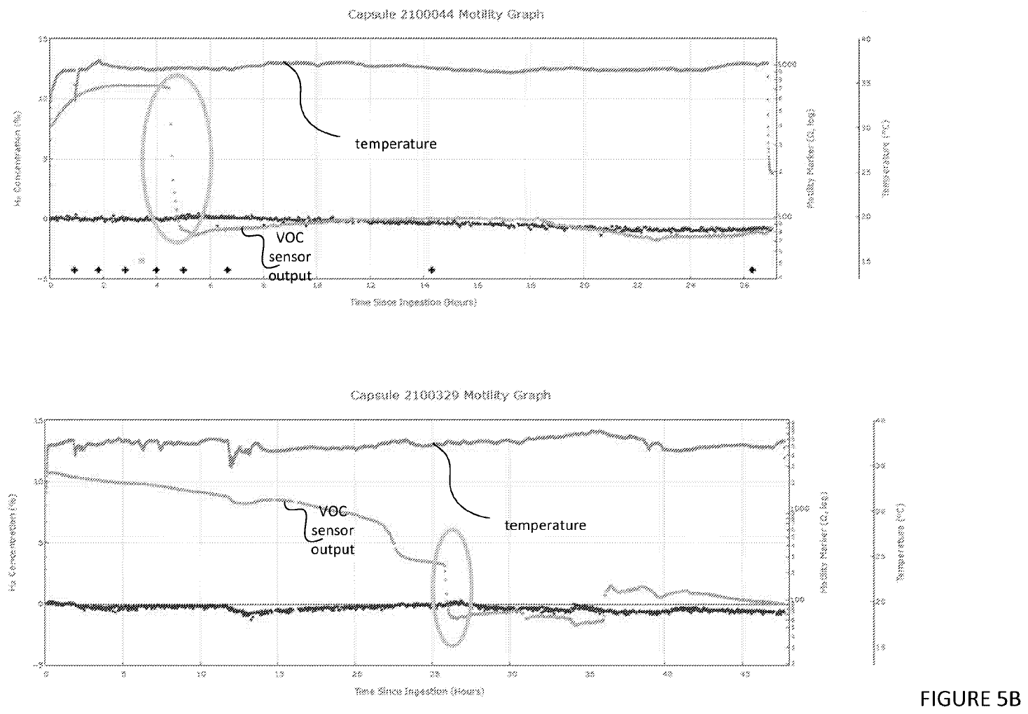

FIG. 5A illustrates VOC output signal obtained in a live implementation of an embodiment;

FIG. 5B illustrates VOC output signal and ICJ indicators in VOC output signal obtained in live implementations of embodiments;

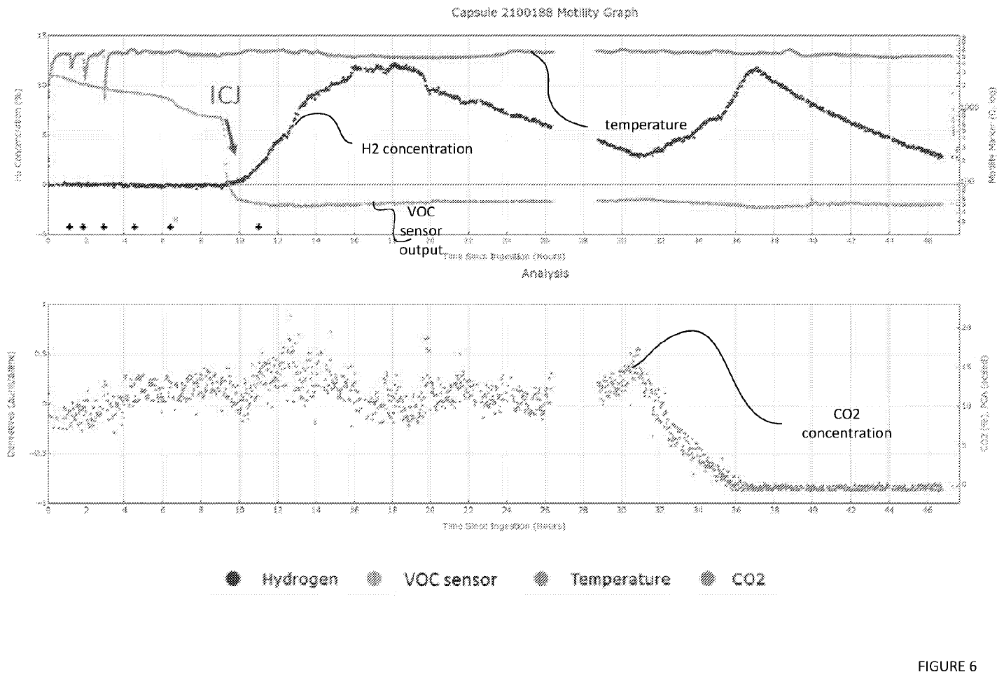

FIG. 6 illustrates VOC output signal and ICJ indicator in VOC output signal obtained in live implementations of embodiments;

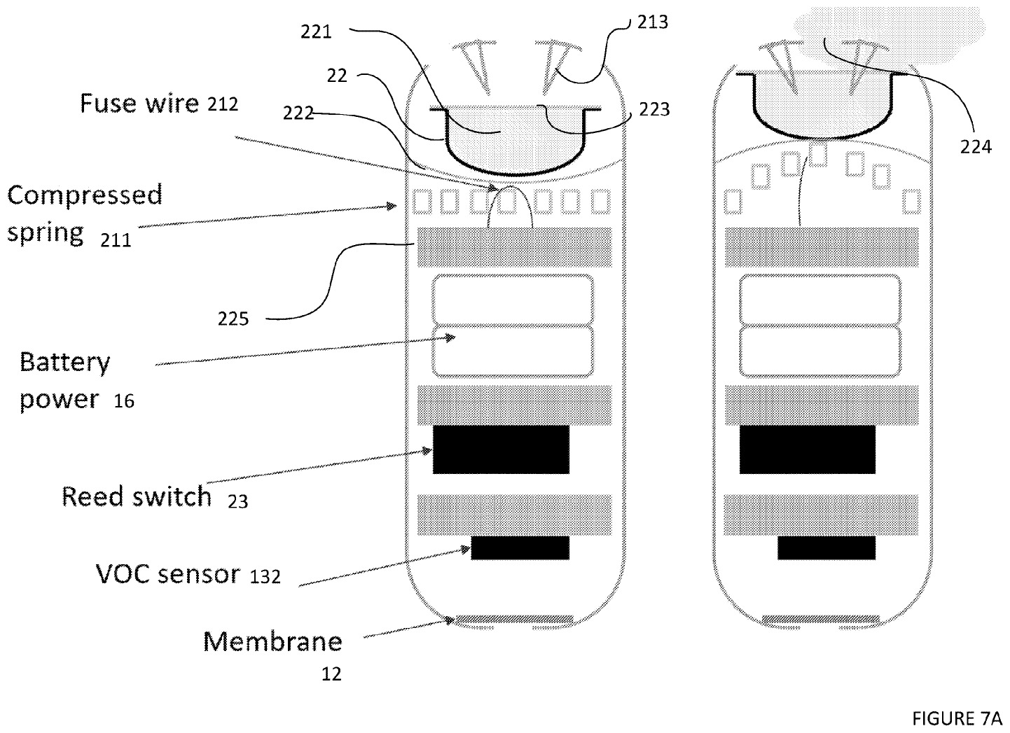

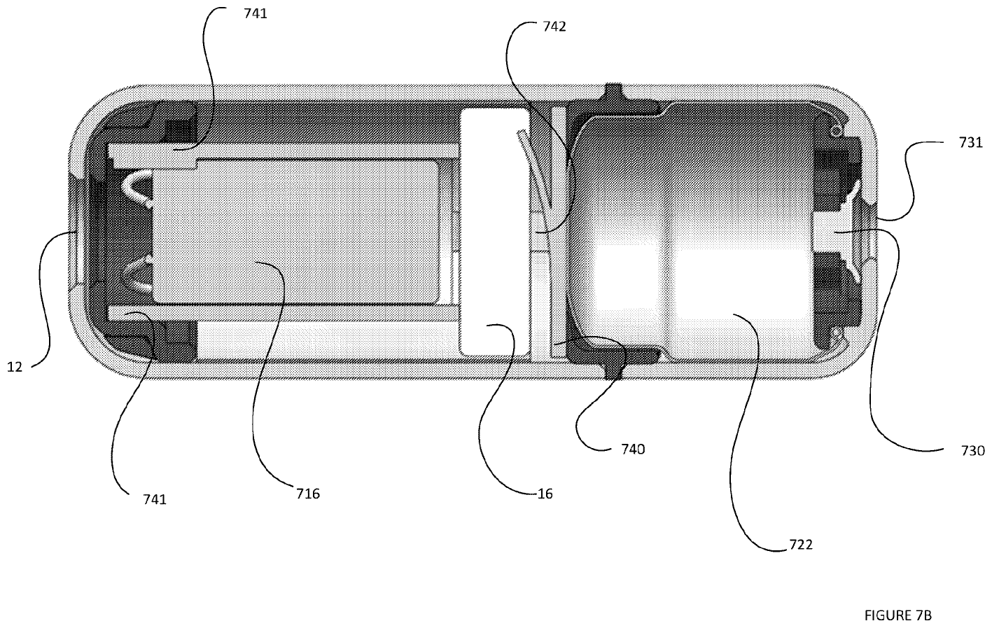

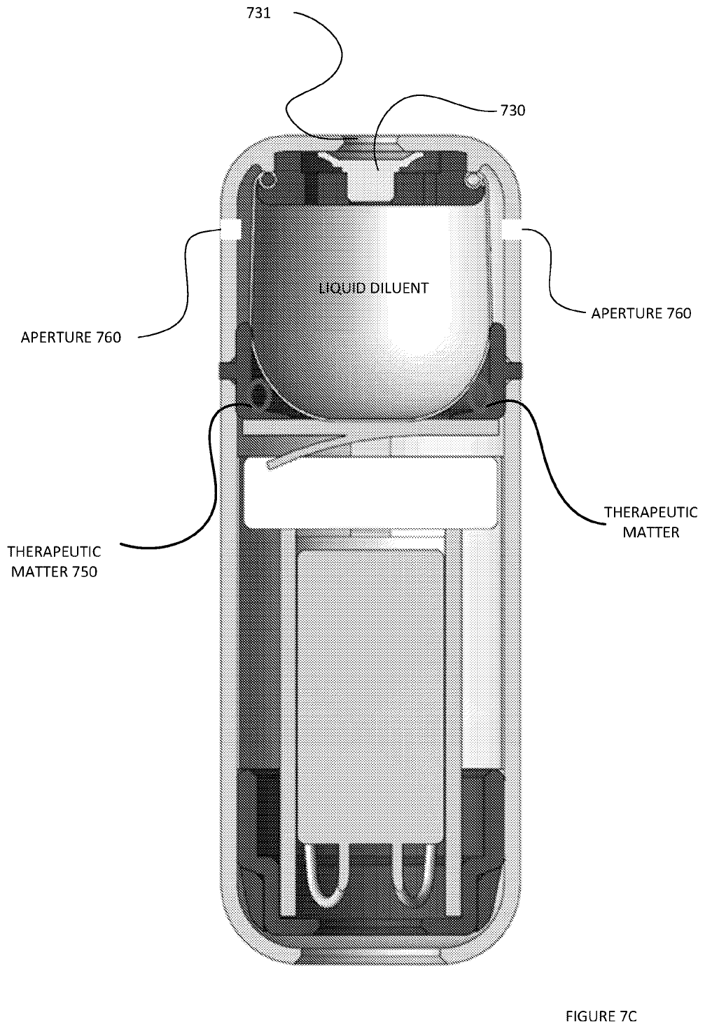

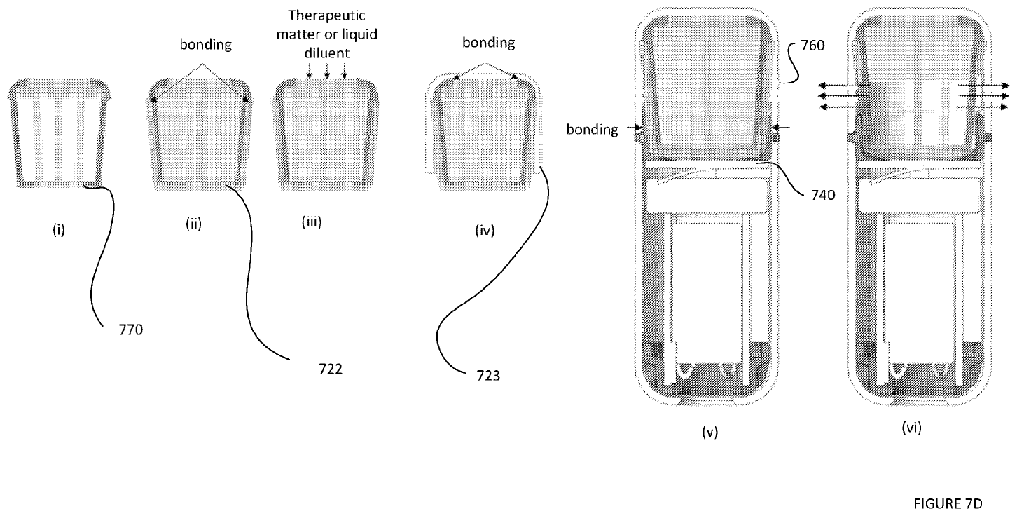

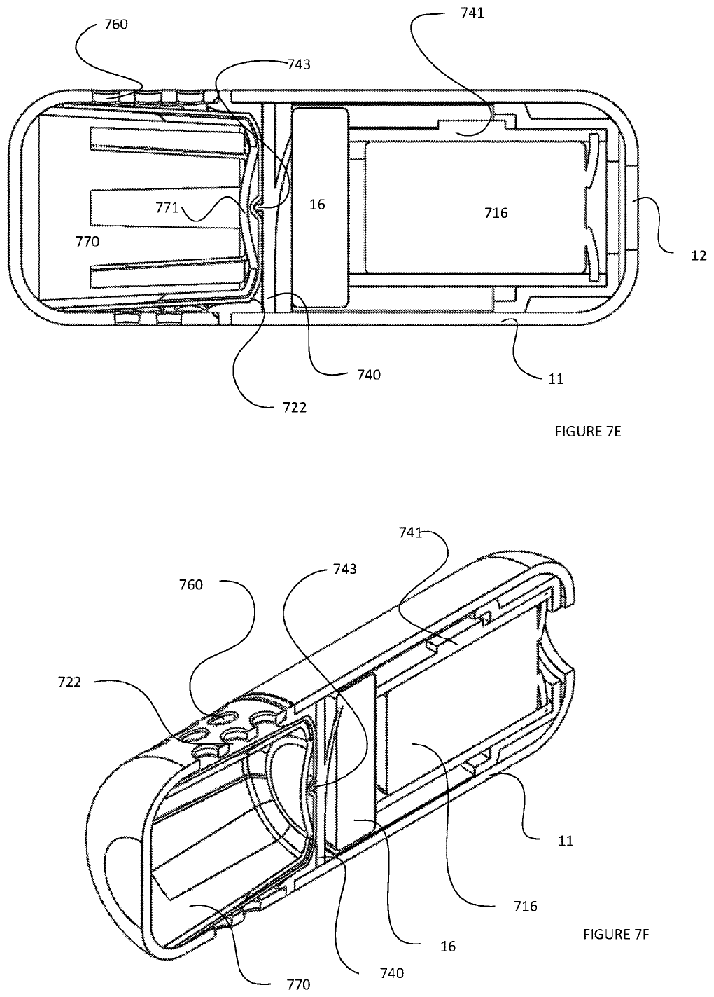

FIG. 7A is a schematic illustration of an ingestible capsule of an embodiment showing release actuator pre- and post-release;

FIG. 7B is a schematic illustration of a release mechanism;

FIG. 7C is a schematic illustration of a release mechanism;

FIG. 7D is a series of illustrations showing formation, filling, and rupturing, of a sealed chamber for therapeutic matter;

FIGS. 7E and 7F are sectional views of an ingestible capsule;

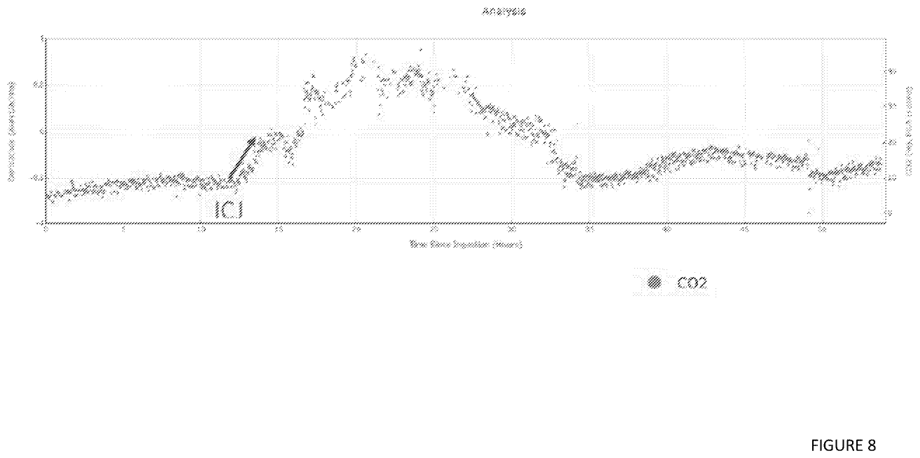

FIG. 8 illustrates CO2 concentration signal and ICJ indicator obtained in a live implementation of an embodiment;

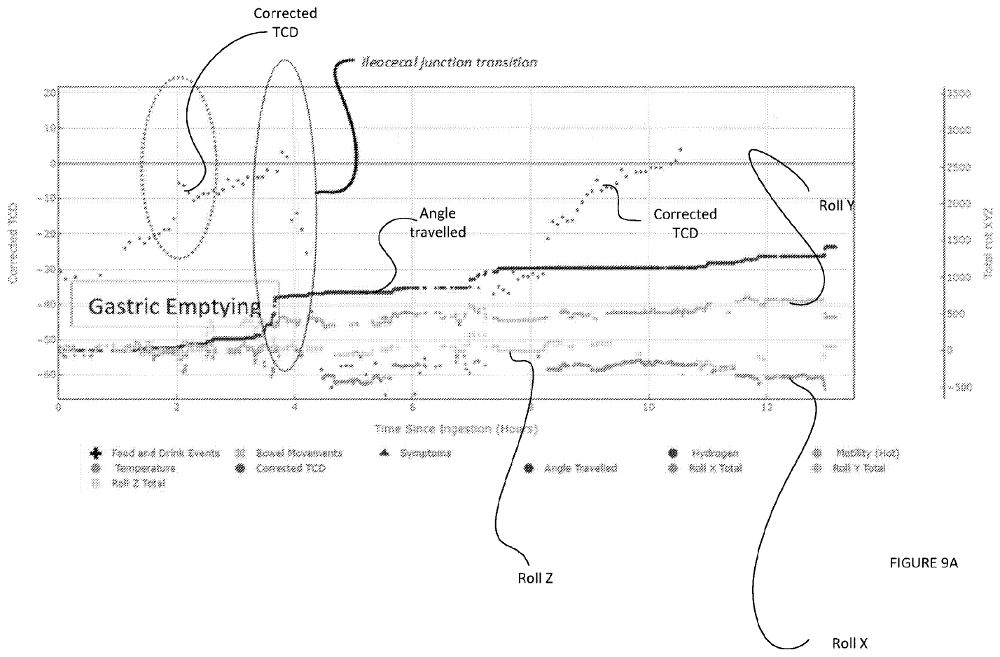

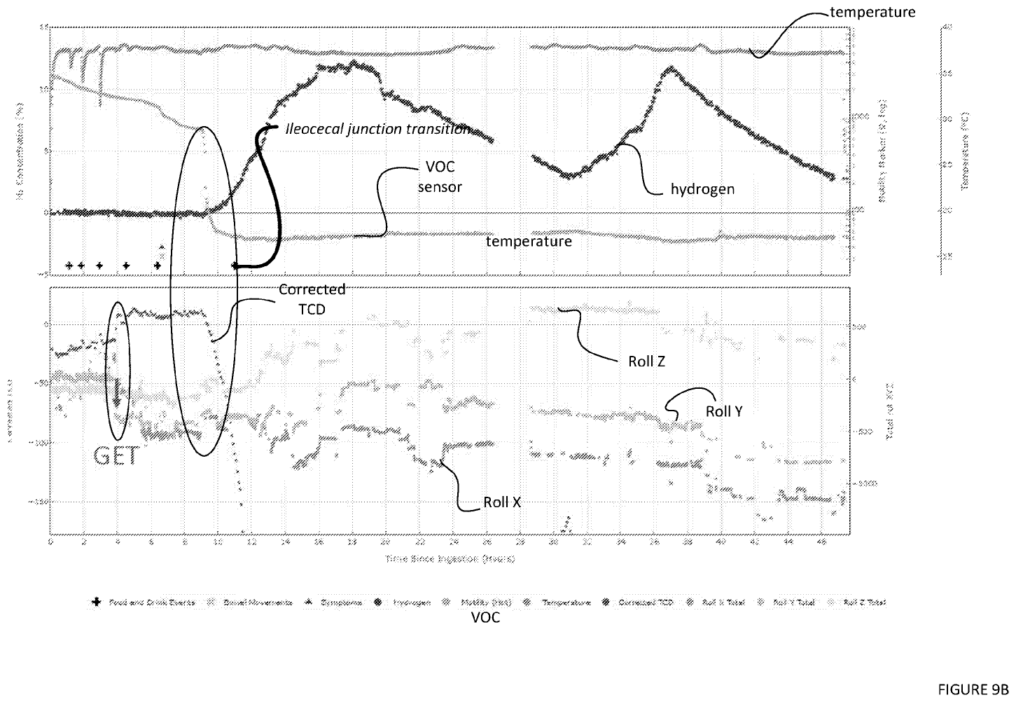

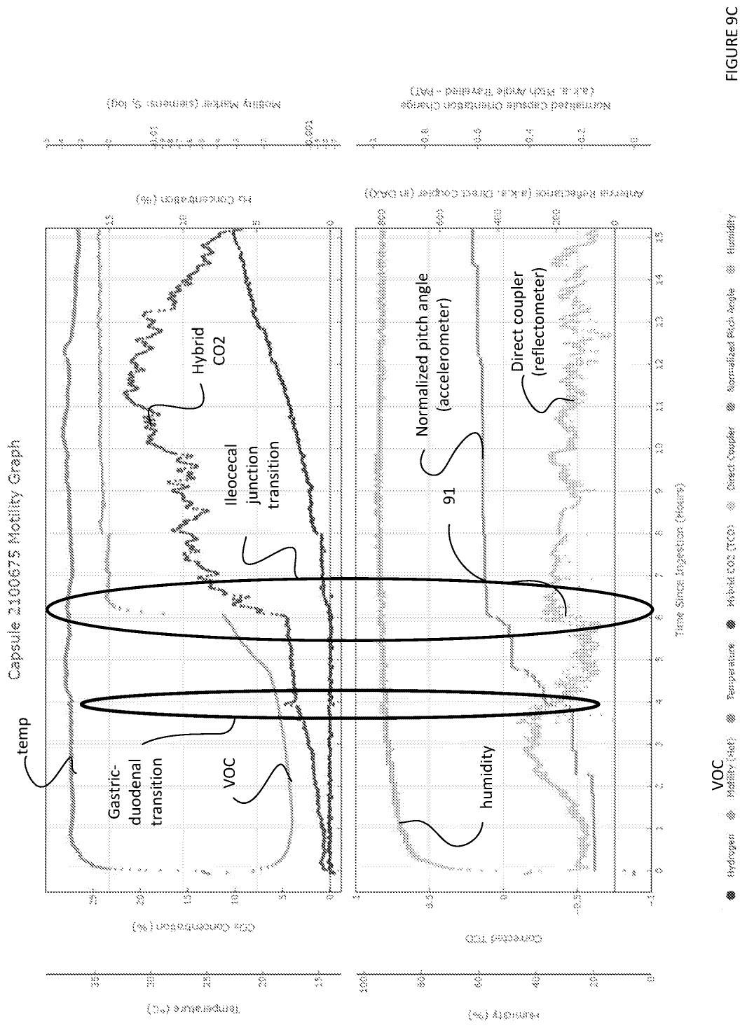

FIGS. 9A to 9C illustrate plots of sensing mechanism output signals from sample ingestible capsules; and

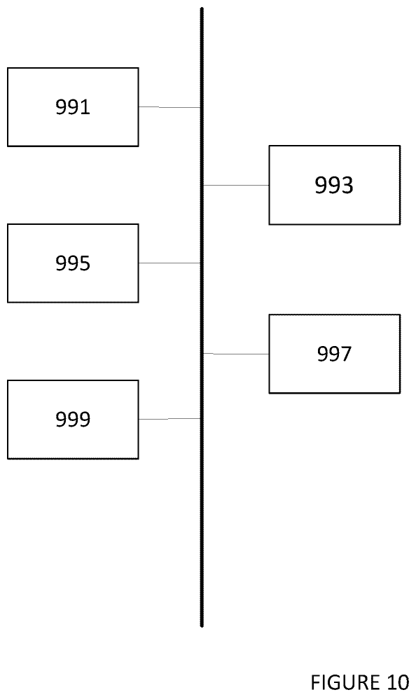

FIG. 10 illustrates a processing apparatus.

### Capsule Structure

FIGS. 1A to 1E are different views of the exterior of an ingestible capsule 10.

As shown in FIGS. 1A to 1E, the ingestible capsule 10 consists of a housing such as a gas impermeable shell 11 which has an opening covered by a gas permeable membrane 12. The gas impermeable shell 11 is formed of a biocompatible indigestible outer layer, such as a biocompatible indigestible polymer. A sensing mechanism is housed within the housing and may include a direct gas sensing mechanism which requires fluid communication with the environment external to the capsule 10 and/or a non-contact sensing mechanism which requires no fluid communication with the environment external to the capsule 10. In capsules 10 comprising a direct gas sensing mechanism which requires fluid communication with the environment surrounding the housing to sense composition of the gas mixture at or surrounding the capsule 10, fluid communication is enabled via the gas permeable membrane 12 illustrated in FIG. 1B. Behind the gas permeable membrane 12 is a sensing headspace housing the direct gas sensing mechanism and sealed off from other components within the capsule 10. That is, any on-board gas sensors are housed within the housing and in fluid communication with the environment surrounding the housing via the gas permeable membrane 12. The remaining components of the ingestible capsule 10 are not in fluid communication with the environment surrounding the housing, until determined release timing at which point some fluid communication is enabled between the external environment and a therapeutic matter carrying compartment 22 via the apertures 760. The apertures 760 are sealed until determined release timing. Since capsule 10 may determine release timing via signal output from a non-contact sensing mechanism, gas permeable membrane 12 is optional.

FIG. 1A illustrates an end of the capsule 10 in which there is an aperture 731 communicating with an aperture in a chamber housing the therapeutic payload which chamber is sealed by a one-way valve or some other sealing member. In the example of the valve, aperture 731 enables access to the valve for insertion of therapeutic matter into the sealed chamber after the capsule 10 has been manufactured, for example via a needle, syringe, or some other mechanism adapted to cooperate with the valve to enable therapeutic matter to flow through the valve into the chamber. It is noted that alternative capsule arrangements do not require an aperture 731 for inserting therapeutic matter to the chamber. In particular, therapeutic matter may be inserted to the chamber prior to completion of the capsule 10, i.e. during manufacture, so that the therapeutic matter is inserted to the chamber, and the sealed chamber is encapsulated in the housing by the bonding of two or more sections of housing to one another. That is, aperture 731 allowing insertion of a needle for inserting therapeutic payload into the capsule 10 is optional.

FIG. 1C is a side view and illustrates the arrangement of apertures 760 in the housing. The apertures enable fluid communication across the housing, however, apertures 760 may be blocked by an elastic material membrane 722 within the therapeutic payload carrying compartment of the capsule 10, said elastic material membrane 722 forming at least a wall of a sealed container carrying the therapeutic payload. The therapeutic matter carrying compartment is a section of the housing and may also include an internal wall dividing the capsule 10 to define a therapeutic payload carrying compartment 22 at the end having the apertures 760 (for example, approximately in line with circumference marked at 112) defined by the internal wall and a section of the capsule housing between the internal wall and the end. The internal wall may be configured to allow electronic connectors to pass across, and to fluidically seal the therapeutic matter carrying compartment of the capsule 10 from a compartment of the capsule carrying the electronics components, for example, including one or more from among power supply, microcontroller, wireless transceiver. Therefore, it is not essential that apertures 760 be blocked. The therapeutic payload is sealed from fluids external to the sealed chamber in which it is carried until the release timing, but fluids from the external environment could be allowed to enter the therapeutic matter carrying compartment 22 of the capsule (defined by the transverse internal wall and the capsule housing between the internal wall and the end of the capsule 10). Reference sign 112 indicates approximate location of internal wall. Capsules may or may not have an external feature at location indicated by 112. Optionally, two portions of capsule housing may be bonded to one another at the circumferential line 112. Optionally, the internal wall may be partially or completely formed by a printed circuit board 740.

FIGS. 1D and 1E are isometric views of the capsule illustrated in FIGS. 1A to 1C.

The housing may be HDPE laser welded with an optional PDMS membrane 12. Optionally, the housing includes one or more apertures 760 through to a therapeutic matter carrying compartment of the interior of the ingestible capsule from the exterior of the ingestible capsule, the said compartment being sealed from a portion of the ingestible capsule 10 housing the electronic components including power supply, sensing mechanism, wireless transceiver, reflectometer, microcontroller, on-board processor, and the apertures 760 being sealed prior to the determined release timing, so that fluids external to the capsule 10 do not mix with therapeutic payload until determined release timing.

One or more of the electronic components may be arranged on a rigid flex printed circuit board. In particular, the antenna may be arranged on a flex printed circuit board, owing to spatial considerations. The flex printed circuit board on which the antenna is printed may be part of, or connected to, a rigid flex printed circuit board on which other of the electronic components are mounted. The capsule may be configured with a rigid only printed circuit board, particularly in a configuration not including an antenna.

Capsule 10 included memory hardware and processor hardware. The memory hardware stores processing instructions for processing output signal from the sensing mechanism to identify indicators of motility events including one or more from among ingestion, gastric-duodenal transition, ileocecal junction transition, and excretion, in order to determine release timing and optionally also to generate report data for transmitting away from the capsule. Alternatively processing instructions may be to coordinate transmission of output signal away from the capsule 10 for processing elsewhere and to receive a signal determining release timing. The memory hardware may also store processing instructions for processing a signal from electrodes or conductive pads in a deployment sensing mechanism for determining that the therapeutic matter has been deployed in order to determine whether to actuate a release mechanism (i.e. in a second or third attempt) and alternatively or additionally to include said determination in report data for transmitting away from the capsule. Processing instructions stored by the memory hardware are executed by the processor hardware. Optionally, the memory hardware and processor hardware are provided as part of a microcontroller. However, it is noted that capsule 10 may be configured with a microcontroller configured to distribute data and power among the various electronic components of the capsule 10, and a dedicated memory hardware and/or a dedicated processor hardware, provided independently of the microcontroller but coupled thereto for exchange of power and data with other components.

Data reported by the ingestible capsule 10 (i.e. data transmission payload transmitted away from the capsule 10 to a receiver apparatus 30) may be used for one or more from among: drug adherence monitoring, dose delivery confirmation, therapy monitoring.

Alternatively, the capsule 10 may be configured without direct gas sensors (wherein direct gas sensor is taken to mean gas sensors requiring direct physical contact between sensor and gas sample). That is, the capsule 10 may be configured with a reflectometer (described in more detail below) and/or an accelerometer instead of the VOC gas sensor, and without a TCD gas sensor, so that contact between sensors and gas mixture or gas sample is not required, and therefore no gas permeable membrane is required. Further, it is noted that TCD gas sensing capability may be provided by a heater side of a VOC gas sensor.

Where a capsule 10 is configured with an accelerometer and to use an output signal generated by an accelerometer, the accelerometer may be a triaxial accelerometer configured to sense direction and magnitude of capsule rotation within the gut, by sensing displacements of a gravity vector with respect to axes fixed in the frame of reference of the capsule. In addition, the accelerometer may be configured to directly measure angular rate of change of the capsule or to perform gyroscopic sensing, as part of the accelerometer sensing. Combining triaxial accelerometry with triaxial angular rate may be referred as 6-axes accelerometer or 6-axes inertial measurement.

The term “non-contact sensing mechanism” is used to refer to either or both of the reflectometer and the accelerometer.

The term “direct gas sensing mechanism” is used to refer to either or both of the VOC gas sensor and the TCD gas sensor.

The term “sensing mechanism” is used to refer collectively to the non-contact sensing mechanism and/or the direct gas sensing mechanism.

It is further noted that the capsule 10 may include one or more direct gas sensors, housed in a compartment defined by the gas permeable membrane and sealed from the remainder of the ingestible capsule content by a gas impermeable membrane.

It is further noted that the capsule 10 may comprise one or more direct gas sensors in addition to the reflectometer and/or accelerometer.

### Capsule Components

FIG. 2 is a schematic of the components inside of the housing. In FIG. 2 the sensing mechanism is a direct gas sensing mechanism provided by the VOC gas sensor 132.

The components include a power source 16, for example, comprising one or more silver oxide batteries. Lithium or other battery chemistries may be used, according to requirements and technical specifications. The power source 16 supplies power to the other components, either via a bus or via direct connections. FIG. 2 is illustrated with a bus connection for illustrative purposes.

The VOC gas sensor 132 is less than several mm in dimension each and the sensor side 132a is sensitive to both O2 and H2 as well as other gases including CH4 and SCFAs. The VOC gas sensor 132 may be considered an aerobic sensor. The VOC gas sensor 132 may be configured to give sensor side readings and driver or heater side readings. The heater side 132b readings may be used to determine thermal conductivity of a surrounding gas and thereby the heater side readings of the VOC are TCD readings. The sensor side 132a readings are used to determine concentrations of volatile organic compounds in the surrounding gases and are VOC readings. The term readings is used as an example of output signal, wherein the output signal represents discrete readings at distinct timings. Alternatively, the output signal may be a continuous signal that is processed as a continuous signal or that is sampled at discrete timings to obtain readings.

The readings from the VOC heater side 132b are used to provide a measurement or indication of H2 concentration in the gas mixture to which the VOC gas sensor 132 is exposed. Optionally, the readings from the VOC heater side 132b are a component signal of the output signal of the VOC gas sensor 132. The VOC sensor side 132a output signal only may be used to identify an ileocecal junction indicator. Alternatively, the VOC heater side 132b output signal may be used in some instances to corroborate an ileocecal junction indicator identified in the VOC sensor side 132a output signal. For example, confidence may be improved by identifying a contemporaneous indicator in the VOC heater side 132b readings.

The VOC sensor side 132a (i.e. the VOC sensing element) may form a resistor in a voltage divider network, the output of which is measured as the VOC sensor side live reading. A transform may be applied at the capsule 10 and/or as part of the processing to transform the output of the voltage divider network into a resistance measurement from the sensing element. The VOC sensor side may be driven with a consistent (i.e. repeated) voltage pulse profile. VOC sensor side readings may be taken in sync with the voltage pulse profile so that there is no phase shift between the voltage pulse and the timing of the readings.

The VOC gas sensors 132 is contained in a portion of the capsule 10 sealed from the power source 16 and other electronic components. The outer surface of this portion of the capsule, or a portion thereof, is composed of a selectively permeable membrane. For example, the VOC gas sensors 132 may include a heater which is driven to heat a sensing portion 132a to temperatures at which sensor readings are obtained (i.e. a measurement temperature). The heater may be driven in pulses so that there is temporal variation in the sensing portion temperature and so that measurement temperatures are obtained for periods sufficient to take readings but without consuming the power that would be required to sustain the measurement temperature continuously. Alternatively, the VOC gas sensor sensing side 132a may be operated “cold”, that is, in the absence of any power being supplied to the heating side. Different implementation scenarios may require different confidence levels in identified indicators and therefore be configured to operate in different manners. Other factors may influence how the VOC gas sensor 132 is operated. For example, if additional payload capacity is required, then running the VOC gas sensor sensing side 132a cold will consume less power and therefore require fewer batteries in the power supply 16. For example, the power supply 16 may be a battery such as a silver oxide or lithium cell battery, or a plurality of such batteries.

Ingestible capsule 10 comprises a microcontroller controlling distribution of data and power among components of the capsule. The microcontroller may itself have data processing capability to identify motility event indicators in the signal output by the sensing mechanism, or there may be a dedicated on-board processor separate from the microcontroller providing such functionality. Alternatively, the output signal may be transmitted via Bluetooth or another data transmission technique to an off-board processor.

Ingestible capsule 10 is configured to use the VOC gas sensor 132 as a direct gas sensing mechanism to gather data in which an ileocecal junction transition indicator and optionally also a gastric-duodenal transition indicator are detectable in order to determine release timing of the release mechanism to release therapeutic matter directly into the GI tract (specifically the small or large intestine) of a subject. Alternatively or additionally to the gas sensor or gas sensors, the ingestible capsule 10 may comprise a non-contact sensing mechanism comprising a reflectometer formed by a transmission antenna 17 in series with a directional coupler 171, and optionally also a tri-axial accelerometer 19, as illustrated in FIG. 3E.

A data transmission payload for transmission to a receiver apparatus 30 by the wireless transceiver 18 may comprise one or more from among: a record of release timing; a record of the one or more identified ileocecal junction transition indicators; a record of the one or more identified gastric-duodenal transition indicators; a record of an electrode signal indicating bursting of a balloon filled with therapeutic payload or liquid diluent; output signal output by the sensing mechanism; and a metric or metrics representing the output signal output by the sensing mechanism. The therapeutic payload in the balloon may be in the form of a liquid or in powder form.

Depending on the regulations of the jurisdiction in which the ingestible capsule 10 is being used, and other considerations, it may be that there is no data transmission payload for transmission away from the capsule 10, so that a wireless transceiver 18 is not required (noting that in some ingestible capsules the wireless transceiver may be required even in the absence of a data transmission payload insofar as it forms part of a reflectometer measuring antenna reflectance signals as part of a non-contact sensing mechanism).

Optionally, ingestible capsules 10 may be provided with a coloured dye with the therapeutic payload, so that identification of the coloured dye in a stool provides evidence of release of the therapeutic payload into the subject GI tract. Coloured dye would also represent the capsule having reached the colon, in which case safe exit of the capsule 10 from the GI tract can be assumed with a high degree of probability. It acts as a low-cost excretion detector. An alternative excretion detector is an on-board temperature sensor and monitoring an output signal therefrom to identify a temperature drop, and transmitting a report of the temperature drop away from the capsule 10 to a receiver apparatus 30 via the wireless transceiver 18 before the capsule 10 is flushed away.

### VOC Gas Sensor

The VOC gas sensor 132 is a semiconductor sensor.

The VOC sensor 132 may be configured to give sensor side readings and driver or heater side readings. The heater side readings may be used to determine thermal conductivity of a surrounding gas and thereby the heater side readings of the VOC are TCD readings. The sensor side readings are used to determine concentrations of volatile organic compounds in the surrounding gases and are VOC readings. Regardless of whether or not heater side readings are being taken, the heater may be driven to heat the sensor side of the VOC gas sensor and the surrounding gas mixture. The heater may be driven in pulses.

The output signal of the VOC gas sensor may be continuous, or may be a series of discrete signal pulses, with each signal pulse representing a sensor reading. In the case of a continuous output signal, a series of readings representing the output signal may be obtained by periodically sampling the output signal.

The VOC sensor side is sensitive to both O2 and H2 as well as other gases and so these readings may be utilised in the second branch. Other gases include CH4 and SCFAs. Optionally, the VOC sensor side readings are not used in the second branch and the VOC sensor side readings are only used to detect an ileocecal junction indicator. Optionally, the VOC sensor side (i.e. the VOC sensing element) forms a resistor in a voltage divider network, the output of which is measured as the VOC sensor side live reading. A transform may be applied at the capsule 10 and/or as part of the processing to transform the output of the voltage divider network into a resistance measurement from the sensing element. The VOC sensor side may be driven with a consistent (i.e. repeated) voltage pulse profile. VOC sensor side readings may be taken in sync with the voltage pulse profile so that there is no phase shift between the voltage pulse and the timing of the readings.

Australian patent application number 2021903378 discloses method, program, and apparatus for determining a location of an ingestible capsule within the GI tract.

### Release Mechanism

The components also include a release mechanism 20. There are three main functional forms of release mechanism 20 disclosed herein. Regardless of functional form, the release mechanism 20 includes a release actuator 21 in physical communication with the therapeutic payload carrying compartment 22. Different release mechanism 20 configurations are illustrated in FIGS. 3A to 3E. Therapeutic payload may be drugs, prebiotics, probiotics, pharmaceuticals, faecal transplant matter, or other therapeutic matter configured for release into the GI tract for therapeutic effect.

The release actuator 21 is physically coupled to the releasable therapeutic payload carrying compartment, and in particular is physically coupled to a portion of the capsule housing at the therapeutic payload carrying compartment so that actuation of the actuator create an opening, aperture, or ruction, in the housing so that the therapeutic payload carrying compartment, and by extension the therapeutic payload itself, is exposed to the environment external to the capsule 10 and thus is released to the GI tract. The electronic components of the capsule 10 are sealed from the therapeutic payload carrying compartment. Alternatively, the capsule housing may contain the therapeutic payload carrying compartment within an open frame structure or via a portion having one or more apertures open to the environment external to the housing, with the therapeutic payload carrying compartment being closed via a valve or some other sealing member that is configured to be opened by the release actuator. For example, a kinked hose that is pulled by the release actuator to release the kink and to deploy the therapeutic payload to the environment external to the capsule 10. The configuration of the release actuator and hose may be such that deployment of the therapeutic payload takes place over a period of time as the capsule 10 transits the colon. For example, the period of time may be between 30 minutes and an hour, between an hour and two hours, or more than two hours.

Particular release actuators are illustrated in FIGS. 7A and 7B, discussed in more detail below. The therapeutic payload carrying compartment 22 may contain a balloon filled with the therapeutic matter, and optionally also further fluid such as liquid or gas or a combination thereof. The release actuator 21 causes the balloon to burst, releasing the therapeutic payload from the balloon and into the subject GI tract via one or more apertures in the capsule housing. It is noted that the portion of the ingestible capsule 10 containing the balloon, and having one or more apertures, is sealed from the portion of the ingestible capsule 10 containing the electronic components (unless a component is specifically described as being in the therapeutic payload carrying compartment 22, for example if required as part of the release actuator 21).

Since it is desirable to minimise power consumption (and thus to minimise size of power source required), the release actuator 21 may store potential energy, such as in a spring, which energy is released by the release actuator 21 in order to create the opening, aperture, perforation or ruction in the housing.

The housing is formed of a biocompatible indigestible polymer. Optionally, the polymer may be scored or otherwise formed to be thinner at the location of the portion physically coupled to the release actuator 21, so that the portion in question may be opened with a high degree of predictability. Furthermore, a surrounding region may be formed thicker, in order to be stronger and thus to reduce likelihood of the opening, aperture, perforation or ruction in the housing extending beyond the coupled portion.

In capsules 10 in which the sensing mechanism is a direct gas sensing mechanism, a headspace is defined in the capsule 10 which is in fluid isolation from the remainder of the capsule interior but is in communication with the GI tract via an aperture in the capsule housing. Within said headspace a perforable sealed (biocompatible, indigestible) film or foil contains a therapeutic payload, wherein the release actuator 21 is configured to perforate (i.e. rupture) the film or foil to release the payload to the GI tract via the aperture in the capsule housing. Noting that the film or foil may be across or partially across the aperture, or may be otherwise in spatial communication with the aperture so that perforation of the foil causes release of the payload into the GI tract via the aperture. The perforation of the foil or film may be by release of a spring pushing the foil or film against a perforating or puncturing member, or may be, for example, by rotation of a rotating member such as a motor that causes a peeling back, a puncturing, or a perforating of the foil or film.

The release actuator 21 may be any actuation mechanism arranged to cause the release of packaged therapeutic payload from the package and into the GI tract. A specific example is illustrated in FIG. 7A and described below.

### Therapeutic Payload Carrying Compartment

The therapeutic payload carrying compartment 22 may comprise a single compartment carrying all (i.e. the required dosage) of the therapeutic payload. Optionally, the therapeutic payload carrying compartment 22 may comprise a plurality of sub-compartments, wherein each sub-compartment carries a portion (i.e. a fraction) of the required dosage of the therapeutic payload. In the case of the plurality of sub-compartments, the capsule 10 may be preset at time of manufacture, or configurable post-manufacture by a clinician (for example by sending a control signal from an external controller to the wireless transceiver 18 which, via the microcontroller 15 or otherwise, switches between two release modes), according to either of two release modes. One being an all-at-once release mode in which the plurality of sub-compartments are released by the release mechanism 21 at a single release timing (i.e. simultaneously or more-or-less simultaneously, such as one after another with no delay between releasing subsequent sub-compartments), and the other being an interval release mode in which the sub-compartments are released by the release mechanism 21 in a series with predefined intervals between consecutive releases. The intervals may be all of a predefined equal length (i.e. regular intervals) or may be of predefined unequal lengths (i.e. irregular intervals). Thus, the release mechanism 21 may be controllable via the microcontroller 15 or otherwise to release the therapeutic matter at a single release timing or at plural distributed release timings.

### Release Mechanism Specific Examples

The first functional form is illustrated in FIG. 3A. In FIG. 3A, the sensing mechanism is a direct gas sensing mechanism, provided by the VOC gas sensor 132. In the first functional form, referred as the local processing form of release mechanism, the release mechanism 20 includes a microcontroller 15, and a determination of release timing is made by processing the output signal of the VOC gas sensor 132 on-board the ingestible capsule 10 by the microcontroller 15. The microcontroller 15 comprises a memory 151 and a processor 152. The microcontroller 15 is configured with midware or software to perform its functionality with respect to identifying an indicator in the output signal of the VOC gas sensor 132 and determining release timing of the therapeutic payload. The line around the release mechanism 20 is dashed to indicate that components therein do not necessarily perform functionality solely relating to the release mechanism 20 and may perform other functions. For example, the microcontroller 15 may perform functions including power and data distribution, data sampling, data processing, motility event indicator identification, release timing determination etc.

The second functional form is illustrated in FIG. 3B. In the second functional form, referred to as the remote processing form of release mechanism, the release mechanism 20 includes a wireless transceiver 18, and a determination of release timing is made by transmitting a representation of the VOC gas sensor output signal to a remote processing apparatus, and receiving a notification signal in response, the notification signal being an instruction from the remote processing apparatus to trigger the actuator 21 to cause the therapeutic payload to be released. FIG. 4 illustrates an arrangement of a system implementing the second functional form (noting that the subject mammal 40 is for illustrative purposes and does not form part of said system), noting that in the particular example of FIG. 4 the remote processing apparatus 22 includes a dedicated remote transceiver 30 for direct communication with the on-board transceiver 18, and a data processor 24 for processing the received data to identify the indicator and thus to determine release timing. The remote processing apparatus 22 may be a single device functioning as transceiver 30 and data processor 24, or may be plural devices cooperating via data communication to implement said functionality.

The wireless transceiver 18 comprises an antenna 17 and directional coupler 171 which in combination function as a reflectometer configured to measure at least amplitude of reflected signals and optionally reflectance and phase of reflected signals. The reflectometer is an example of a non-contact sensing mechanism which may be used in combination with or in place of the direct gas sensing mechanism 132.

In addition, ingestible capsules 10 may implement both the local processing functional form and the remote processing functional form. The third functional form is illustrated in FIG. 3C. The third functional form combines local processing and remote processing to process the output signal of the VOC gas sensor 132 and determine release timing.

Therefore, a system implementing the third functional form may have the arrangement illustrated in FIG. 4 (noting that the subject mammal 40 is for illustrative purposes and does not form part of said system).

### NON-Contact Sensing Mechanism

FIG. 3E is a schematic of components in an ingestible capsule 10 in which the sensing mechanism is a non-contact sensing mechanism, provided by at least one from among an accelerometer 19 such as a triaxial accelerometer, and a reflectometer formed by the antenna 17 and directional coupler 171. The microcontroller 15 samples measurements of reflectance from the antenna 17 via the directional coupler 171. The signal being transmitted may be a data signal having data transmission payload either for reporting information away from the ingestible capsule 10 (such as one or more from among motility event timing, release timing, indication that release has occurred, excretion event timing, etc) or may be a pilot or other signal with no data payload and transmitted by the wireless transceiver 18 solely for the purpose of making reflectance measurements. Reflectometer may be configured to measure only amplitude or may be configured to measure phase and amplitude of reflected signal. Reflectometer may be configured to measure reflectance from a signal transmitted at a particular carrier frequency or frequencies. Reflectometer may be configured to measure reflectance from a signal transmitted at carrier frequencies sweeping between two defined extremes at predefined intervals therebetween.

Readings from the reflectometer (amplitude measurements) are shown in FIG. 9C and illustrate presence of a gradient change at ileocecal junction transition timing, and also at gastric-duodenal transition timing. Therefore release timing may be determined based on readings of the reflectometer.

Furthermore, the accelerometer readings of FIGS. 9A to 9C, and the metrics calculated from the raw accelerometer readings, illustrate presence of indicators at ileocecal junction transition timing, and also at gastric-duodenal transition timing. Therefore release timing may be determined based on readings of the accelerometer.

Capsules 10 may comprise only one of reflectometer and accelerometer. Since reflectometer has functionality beyond sensing, ie. reporting and other communication with receiver apparatus 30 or equivalent, ingestible capsules with reflectometer as the only sensing mechanism are particularly cost effective.

### Non-Contact Sensing Mechanism: Reflectometer and Accelerometer

Optionally, the sensing mechanism may be a non-contact sensing mechanism comprising both the accelerometer and the reflectometer.

If the capsule 10 is configured to release the therapeutic payload into the small intestine, release timing may be determined by identifying a gastric duodenal transition indicator in the reflectometer readings and a contemporaneous (to within a predefined time window) gastric duodenal transition indicator in the accelerometer readings. Release timing may be immediate or at a predefined delay after indicator timing.

If the capsule 10 is configured to release the therapeutic payload into the large intestine, release timing may be determined by identifying an ileocecal junction transition indicator in the reflectometer readings and a contemporaneous (to within a predefined time window) ileocecal junction transition indicator in the accelerometer readings. Release timing may be immediate or at a predefined delay after indicator timing. Optionally, a further condition may be that, at a timing preceding the ileocecal junction transition indicators, a gastric-duodenal transition indicator has been detected in either or both of the reflectometer readings and the accelerometer readings. Optionally, processing to detect ileocecal junction transition indicators may not begin until the gastric duodenal transition indicator or (contemporaneous to within a predefined time window) indicators are detected.

### System Architecture

As shown in FIG. 4, a system, in addition to the capsule 10, further comprises a receiver apparatus 30 which receives data transmitted by the capsule from within the GI tract of the subject mammal during the live phase. Concurrently or subsequently, the receiver apparatus 30 processes the received data and may also upload some or all of the received data to a remote processing apparatus 24 such as a cloud-based service for further processing. The remote computer may be a cloud resource, or may be a standalone computer at a clinician premise at which the subject is a patient, or may be a server (be it cloud-based or otherwise) at a service provider to which the clinician is a subscriber/customer/servicer user.

The receiver apparatus 30 may be a dedicated device (designed for storing and optionally processing data received from the capsule 10) or may be a general purpose device such as a smartphone. The smartphone may be running an app (downloaded thereto in advance of capsule ingestion) for storing and optionally processing data received from the capsule 10. In particular, the capsule 10 may comprise a Bluetooth transceiver 18 and the receiver apparatus 30 may be a Bluetooth-enabled tablet, smartphone, or personal computer.

Optionally, a system may further comprise a remote processing apparatus 24 such as a server forming part of a cloud computing environment or some other distributed processing environment. The remote processing apparatus 24 may be a server provided by or on behalf of a clinical centre at which subject 40 is a patient and taking responsibility for interpreting the results generated by the capsule 10 (i.e. the data transmission payload) and reporting them to the subject 40.

Since processing may be performed by the smartphone application or dedicated receiver device 30, the term remote processing apparatus may also be used as a collective referring to either or both of the receiver apparatus 30 (whether that be smartphone or dedicated device) and computing apparatus 24.

### Power Source

Power source 16 is a battery or super capacitor that can supply the power for the sensors and electronic circuits including the processor hardware 151 and memory hardware 152. A life time of at least 48 hours may be set as a minimum requirement for digestive tract capsules. A number of silver oxide batteries in the power source 16 is configurable, depending on the needed life time and other specifications for the capsule. For example, long-range Bluetooth may consume more power than standard Bluetooth. Capsules may be configured to switch from long-range Bluetooth transmission to standard Bluetooth transmission once the stored energy in the battery (or batteries) drops below a predefined threshold, wherein the on-board processor is configured to monitor stored energy level. Capsule 10 requires sufficient stored energy to actuate the release mechanism.

In a specific example, the capsule 10 includes a super-capacitor which is trickle charged from a silver oxide battery or another type of battery and then releases its stored energy into a wire in contact with a balloon containing the therapeutic matter, thus heating the wire, bursting the balloon, and releasing the therapeutic matter into the GI tract via apertures in the capsule housing.

### Communication Between Capsule and Receiver Apparatus

During a live phase of the capsule 10 (i.e. following an initiation or other power-on event) connectivity between the capsule 10 and the receiver apparatus 30 is via the data transmitter on the capsule, which may be part of a wireless transceiver 18, for example a Bluetooth transceiver, which may operate according to a standard Bluetooth transmission protocol or according to Bluetooth Low Energy transmission protocol. Other operable communication technologies include LoRa, wifi and 433 MHz radio. Other radio frequencies could be used in addition to or instead of 433 MHz.

Optionally, there may be plural wireless transceivers on the capsule 10, such as a Bluetooth, or primary, transceiver and an NFC, or secondary, transceiver. A single integrated chip may provide both, so that certain of the circuitry need not be duplicated. However, the single integrated chip includes two separate antennae, one for Bluetooth communication (for example at 2.4 Ghz) and one for NFC communication (for example at 13.6 MHz). The capsule 10 may comprise two separate wireless communications mechanisms, each being configured to at least one of send and receive data, from a smartphone or tablet within communication range. The wireless communication mechanisms may be a primary, or Bluetooth, wireless communication mechanism and a secondary, or NFC, wireless communication mechanism.

For example, the smartphone or tablet 30 may be running an application managing data communication with the capsule 10 and in particular configured to at least one of store, process, and transmit data from the capsule 10. The same application may facilitate communication with the communication mechanisms on the capsule 10, for example, by encoding one or more signals transmitted from the smartphone or tablet with a code which the capsule 10 has been preconfigured to accept as a key with which to unlock functionality. That is, the capsule 10 has been preconfigured only to respond to received signals encoded with the code. The code is unique to the specific capsule instance.

### Pairing or Coupling

The primary wireless data transmitter may be a Bluetooth transmitter, a wifi transmitter, a radio transmitter, or another form of wireless data transmitter. A primary radio transmitter may be configured to transmit in the 433 MHz band. In any case, the primary wireless data transmitter may be provided as part of a primary wireless data transceiver. For example, the primary wireless data transceiver may receive signals at least in performing pairing or any other form of coupling to a recipient device 30. The capsule 10 may be configured to enter into a wireless pairing or coupling mode immediately upon initiation of the live phase (i.e. first full power-on), wherein a subject or another user is instructed (via written instructions or via an application running on the recipient device itself) to pair or couple the capsule 10 and specifically the primary wireless transceiver thereof to the recipient device 30 prior to ingestion of the capsule 10. However, embodiments may be configured such that pairing or coupling is not necessary, for example the capsule 10 may be configured to broadcast data to a recipient device in a data transmission technique that is agnostic to pairing or coupling status. Pairing or coupling establishes a data communication connection or pathway for transmission of the data transmission payload from the capsule 10 to a receiver apparatus 30 by the primary data transceiver.

Data transmission payload is data to be transmitted away from the capsule 10 to the receiver apparatus 30, either in pre-excretion transmission routine, post-excretion transmission routine, or both. Data transmission payload may comprise one or more from among: raw readings from the sensors or quasi-sensors on board the capsule 10; a metric or metrics calculated by on-board processing of said raw readings; a motility event indicator identified by on-board processing of said raw readings; report data comprising an indication of therapeutic matter release and timing thereof.

### Activation via NFC

In a specific example, the capsule 10 may comprise a Bluetooth transceiver for transmitting readings, event reports, and/or other payload data to a Bluetooth transceiver of the receiver apparatus 30, and an NFC transceiver for performing a handshake or equivalent with an NFC transceiver of the receiver apparatus 30 in order to power on the capsule 10.

NFC could be used for activation independent of having a bluetooth transceiver. BT runs at 2.4 GHz while NFC is at 13.56 MHz, so they may utilise separate antennas.

Optionally, the secondary transceiver may be configured to receive an encoded activation control signal from a smartphone or tablet running an application configured for managing interactions between the smartphone or tablet (tablet in this context meaning tablet computer) and the capsule 10, which encoded activation control signal initiates a live phase of the capsule 10 during which capsule sensors take readings and the readings themselves or metrics and/or reports based on the readings are transmitted from the capsule 10 to the smartphone or table via the primary wireless data transceiver. Thus, the secondary wireless data transceiver is active in a listening phase which precedes a live phase of the capsule. The primary wireless data transceiver is inactive (i.e. consuming no power whatsoever) during the listening phase. Once the encoded activation control signal is received (and the capsule 10 powered on in response) the listening phase ends and the secondary wireless data transceiver becomes inactive. The primary wireless data transceiver is active during the live phase.

In order to conserve battery power, capsule 10 operates in a standby or listening mode during the time between release from manufacturing and initiation of the live phase during which readings are recorded by the on-board sensors and transmitted away from the capsule. The standby or listening mode is an extremely low power mode. A live phase of the capsule is initiated prior to ingestion by the subject mammal. A mechanism for ending the standby or listening mode and entering a live phase include a reed switch coupled to a magnet on the packaging which is triggered by release of the capsule from the packaging and when triggered powers on the processor, sensors, and primary transceiver (i.e. initiates the live phase). An alternative mechanism is based on Near Field Communication, NFC. In the alternative mechanism, the capsule 10 is maintained in the standby or listening mode (which in the particular example of the NFC is a SENSE mode) prior to being issued to the subject. In the listening mode, when an electromagnetic field is detected with an appropriately encoded activation control message, the on-board microcontroller (i.e. processor) enters a live phase. A tablet computer or mobile phone running an application configured for the purpose of managing interactions with the capsule 10 and the processing of data received therefrom, and having NFC capability, can generate the appropriately encoded activation control message. In particular, a back-end server may link a user account to a particular capsule instance, so that when that user is logged in to the application and selects to activate a capsule, the application performs a lookup to the back-end server to determine how to encode the activation control message. In other words, optionally the encoding is unique per capsule. Alternatively, the encoding may be uniform across a batch of capsules or all capsules.

The NFC transceiver may be on the same integrated chip as the primary transceiver. The NFC transceiver may be positioned at an end of the capsule and close to the housing, to facilitate communication with the tablet computer or mobile phone.

### Primary Transceiver: Post-Excretion Data Transmission Routine

There are two principal data transmission routines, which ingestible capsules may be configured to use either or both of, depending on implementation details (i.e. use case). In a post-excretion data transmission model, signals from the sensors are received at the processor hardware 151 (utilising also the storage capabilities of the memory hardware 152) and processed on-board the capsule 10 in order to determine release timing of the release mechanism. The on-board processor 151 may also be configured to compile and store as data transmission payload pending transmission to the receiver apparatus 30 a report of the therapeutic payload release, wherein said report may comprise one or more from among: a record of the indicator, readings, or metric triggering the release mechanism, a record of release mechanism trigger and timing thereof, an indicator of successful actuation of the release mechanism. Other characteristics and readings or groups of readings of interest may include, for example, maximum or minimum readings from specific sensors or from metrics calculated by combining sensors. The maximum or minimum readings may be local maximum or local minimum readings, wherein local is defined by, for example, predefined timings or motility events determined to have occurred by the capsule 10 itself. A specific example is maximum or minimum H2 concentration, which is a metric calculated from the gas sensor readings by an appropriately calibrated processor hardware.

For example, data transmission payload may comprise one or more from among: a record of the indicator, readings, or metric triggering the release mechanism, a record of release mechanism trigger and timing thereof, an indicator of successful actuation of the release mechanism.

Capsules 10 may be arranged with no data transmission mechanism or with only an NFC transceiver for activation purposes: the transmitting data transmission payload away from the capsule 10 is optional. For example, the on-board processor may process sensor readings to determine release timing of the release mechanism, and actuate the release mechanism accordingly so that the capsule 10 releases its therapeutic payload. Further processing and data transmission steps are optional. Reporting data may be stored and transmitted away from the capsule 10 in embodiments configured accordingly, or no such storage and transmission is performed.

Capsules 10 which do not store and transmit data away from the capsule 10 may still be equipped with a transmission antenna, since the transmission antenna may form part of a reflectometer which operates as a sensor sensing dielectric properties of the environment surrounding the capsule 10 and therefore indicative of the location of the capsule 10 within the GI tract. In such a case, the transmission antenna may be configured to frequency sweep or transmit at a fixed frequency, using, for example, a pilot signal with no data payload.

In the post-excretion data transmission routine, the data transmission payload is transmitted by the wireless transceiver once excretion of the capsule 10 from the GI tract is detected (for example by the temperature sensor 14a signal and/or by the accelerometer 19 signal). Metrics further include peak H2 level or value, timing of peak H2, and total H2 (area under the curve). Such metrics may be calculated by the on-board processor hardware 151 during passage through the GI tract of the subject, and transmitted away from the capsule 10 to a receiver device in post-excretion transmission as part of a report or otherwise.

In the post-excretion data transmission routine, the transmission may be via a Bluetooth transmission mode that is not dependent upon pairing status. That is, for example, if the Bluetooth transceiver is paired to a receiver apparatus 30 then it transmits the data transmission payload to the paired receiver device, and if the Bluetooth transceiver is unpaired then it broadcasts the data transmission payload to a recipient device 30 in the absence of pairing in an inquiry mode (which may be referred to as discovery mode or beacon mode). Bluetooth protocol has an inquiry mode in which a device broadcasts a unique identifier, name and other information. The data transmission payload, or part thereof, may comprise or be included in the said other information. In particular, the data transmission payload may be prioritised or otherwise filtered by the processor hardware 151 so that information deemed particular important such as an indication that excretion has occurred (it is important for clinical reasons to know that the capsule 10 has been excreted) and potentially information such as timing of release of thereapeutic payload via release mechanism, is transferred away from the capsule 10 in preference to other information.

Following the inquiry mode transmission, the transceiver may again attempt to pair, connect, or otherwise couple, with the recipient device, and if successful, to transmit the remainder of the data transmission payload. Of course, said pairing, connecting, or coupling, may have been performed initially pre-ingestion so that post-excretion the Bluetooth transceiver is attempting to re-pair, re-connect, or re-couple, with the receiver device 30. It is noted that the present discussion uses Bluetooth as an example of a transmission protocol, but that the same techniques could be applied to different transmission protocols.

In the event that there is data transmission payload pending transmission away from the capsule 10 after the broadcast of the unique identifier, name, and other information during the Bluetooth inquiry mode, then capsule 10 may be configured to initiate or re-initiate a data communication connection (i.e. a pairing or re-pairing) with a receiver device 30. Upon successful initiation or re-initiation of the communication connection, transmission of the said data transmission payload pending transmission away from the capsule 10 is performed whilst the data communication connection remains active.

The Bluetooth, primary, transceiver 18, or any other primary wireless data transceiver 18, may be configured to automatically re-connect following an initial (i.e. pre-ingestion) connection to a receiver device 30. The receiver device 30 may run an app or web app to guide the subject in terms of how to ingest the capsule 10, to notify the subject that the excretion event has been determined, and optionally also that the data transmission payload has been successfully transmitted to the receiver device 30 and so the capsule 10 may be flushed away. It is noted that the terms pair, connect, and couple, are interchangeable in the present document, each representing the establishment of a wireless connection between two devices for wireless data transfer.

It is noted that data transmission payload may be being transmitted throughout passage of the capsule 10 through the GI tract, dependent upon pairing, coupling, or connection to the receiver device 30. However, confirmation that therapeutic payload has been released and that occurrence of an excretion event has been determined by the capsule is information that is of particular importance since safety of capsule 10 is reliant on the capsule 10 being excreted, and efficacy is reliant upon release of therapeutic payload. Therefore, information representing release of therapeutic matter and timing thereof, and determination of occurrence of the excretion event (i.e. a report thereof) is prioritised and may be transmitted in a broadcast or inquiry mode, whereas the remaining data transmission payload (should there be any) is transmitted once connection between the wireless data transmitter 18 and the receiver device 30 is established.

In Bluetooth inquiry mode, data can be transmitted to the receiver apparatus 30, or to any Bluetooth receiver apparatus within range of the capsule 10, without pairing. The primary transceiver 18 is operable in a Bluetooth inquiry mode or a Bluetooth low energy mode. Capsules 10 may store and transmit among the data transmission payload readings from one or more sensors representing a predefined period either side of the identified motility indicators. For example, gas sensor signals only, or for all sensors. Such readings may be used to add confidence to the identified motility indicators in terms of determining whether or not a motility event has occurred, and/or may provide other information useful in a health or clinical context.

More generally, data transmitted according to the post-excretion data transmission routine may be any of the data transmission payload that has not already been transmitted. For example, the primary transceiver 18 may be configured to transmit the data transmission payload to a paired receiver apparatus while still in the GI tract (this element of the transmission is referred to herein as pre-excretion data transmission routine). However, owing to issues such as signal attenuation, noise, power supply issues, temporary pairing failure, or if pairing was never performed in the first place, or for any other reason, some or all of the data transmission payload may be pending transmission at the point of excretion. In that case, the remaining data transmission payload is transmitted according to the post-excretion data transmission routine once excretion is detected. It is noted that down-sampling of the data transmission payload may be performed prior to transmission via the post-excretion data transmission routine. Furthermore it is noted that some elements of the data transmission payload may be prevented from transmission via the post-excretion data transmission routine. For example, since bandwidth, and also time within which to transmit, may be limited, it may be that the report data including determined release timing of therapeutic matter, confirmation of release of therapeutic matter, indication of occurrence of excretion event, themselves are included, but that sensor readings are excluded from the data to be transmitted according to the post-excretion data transmission routine.

### Primary Transceiver: Post-Excretion Data Transmission Routine

In a pre-excretion data transmission technique, the signals may be transmitted continuously (errors, faults, and other unintentional interruptions notwithstanding) by the primary transceiver 18, such as sensor readings or metrics representing sensor readings. Alternatively, event reports may be transmitted in real-time, such as motility events (passage between sections of GI tract, ingestion, excretion), and/or therapeutic matter release event and timing thereof. In the pre-excretion data transmission routine, the processor hardware 151 coordinates the receipt of the signals from the sensors and the storage at the memory hardware 152 for transmission by the wireless transceiver 18.

In the example of a Bluetooth primary transceiver 18, in the pre-excretion transmission routine the transceiver may be operated according to a long-range or coded-phy Bluetooth transmission procedure, such as BTLE Coded PHY. A signal power enhancement of around 10 dB is achievable via BTLE Coded PHY Bluetooth transmission procedure, relative to a conventional Bluetooth transmission protocol.

During a data transmission phase of the ingestible capsule 10 (i.e. which in the post-excretion data transmission routine is in a short burst post-excretion and in the pre-excretion data transmission routine is during passage through the GI tract while the ingestible capsule 10 is in use, that is, in the GI tract of a subject mammal 40 and obtaining and transmitting readings and/or report data) the wireless transmitter 18 transmits the readings to a receiver apparatus 30, which may be a dedicated device for receiving and storing the readings (and optionally with a user interface) or may be a multi-function device such as a mobile phone (such as a smart phone) with a Bluetooth transceiver or another wireless transceiver. The mobile phone may be running an application which processes some or all of the data transmission payload to generate a motility report or diagnosis of a medical condition based on motility indicators, or a report of therapeutic payload delivery and/or excretion, and/or diagnostic indicators either included in the data transmission payload or derivable therefrom. Alternatively, the application may be configured to transmit the data transmission payload to a server or another processing apparatus to generate a report or diagnosis based on the data transmission payload. The subject mammal 40 need not remain within a specific range of the remote computer 20 during the live phase.

Capsules 10 equipped with a Bluetooth transceiver 18 may communicate directly with a smartphone of a user, which obviates any need for a dedicated receiver apparatus (the smartphone taking on the role of receiver apparatus 30). The receiver apparatus 30 (whether a dedicated device or a mobile phone or tablet computer) may process the readings or other received data itself or may upload the received data to a remote computer 20 for processing (i.e. identifying motility indicators, determining motility event timings, reporting therapeutic matter release and excretion, resolving gas analytes). The upload may be continuous during a live phase of the capsule, or the upload may be after the live phase of the capsule is terminated. The receiver apparatus 30 may also store the readings, so that loss of connectivity between the receiver apparatus 30 and a remote processing apparatus is not critical.

### Pre-Processing

The on-board processor 151 may apply one or more processing or pre-processing steps, as discussed in more detail below. Digitisation of the readings is performed either by the sensors themselves, by the processor 151 or by the wireless transceiver 18. The digitised readings are transmitted via the antenna 17. The readings of the capsule 10 are made at an instant in time and are associated with the instant in time at which they are made. For example, a time stamp may be associated with the readings by the microcontroller 15, the wireless transmitter 18, or at the receiver apparatus 30 or remote computer 20. For example, if readings are made and transmitted more-or-less instantaneously (i.e. within one second or a few seconds) by the wireless transmitter 18 then the time of receipt by the receiver apparatus may be associated with the readings as a time stamp. Processing of the readings discussed further below is somewhat dependent on the relative timings of the readings (i.e. so that contemporaneous readings from the different sensors can be identified as contemporaneous), however accuracy to the level of one second, a few seconds, or 10 seconds, is sufficient.

### Primary Transceiver Configuration and Antenna Reflectance Related Readings

Commercial bands (such as 433 MHz and Bluetooth 2.4 GHz) are used by the antenna 17 of the primary transceiver as electromagnetic waves in this frequency range can safely penetrate the mammalian tissues 40. Other commercial bands may be used. Coding may be applied at the digitisation stage to ensure that the data transmitted by the capsule 10 is distinguishable from data transmitted by other similar capsules 10. The transmission antenna 17 may be, for example, a pseudo patch type for transmitting data to the outside of the body data acquisition system. Power source 16 is a battery or super capacitor that can supply the power for the sensors and electronic circuits. Power source is selected to have enough stored energy to power the capsule 10 from ingestion until excretion, taking into consideration power required to actuate the therapeutic matter release mechanism, to take readings required to determine release timing, and if the capsule 10 is configured to transmit signals away from the capsule 10, to transmit those signals, as well as powering a processor and/or microcontroller. A number and capacity of silver oxide batteries in the power source 16 is configurable, depending on the needed life time and other specifications for the capsule.

Readings of reflectance from the antenna may be used as an indicator of GI tract location, either in combination with readings from other sensors or in isolation. Such readings may be obtained as reflectance from signals transmitted from the antenna and comprising a data transmission payload, or may be from signals having no data transmission payload and transmitted solely for the purpose of obtaining reflectance readings. In the case of signals comprising data transmission payload, the frequency may be determined according to transmission protocol, in the case of no data transmission payload, the frequency may be a fixed pilot signal frequency, or the antenna may be configured to frequency sweep periodically.

The antenna 17 may be in series with a directional coupler 171. The directional coupler 171 and the antenna 17 are configured as a reflectometer. The reflectometer measures the amplitude of reflected signals by means of a diode detector. The amplitude measurements of the reflectometer are readings that represent electromagnetic properties of material in the vicinity of the capsule (in particular at the interface between capsule housing and GI tract). For example, good impedance matching between the antenna and the environment surrounding the capsule 10 will result in a low amplitude reflectance signal and therefore low amplitude measurement. Poor impedance matching between the antenna and the environment surrounding the capsule 10 will result in a high amplitude reflectance signal and therefore high amplitude measurement.

A reflectometer formed by the antenna 17 and directional coupler 171 measures amplitude of reflected signal at the antenna 17 of the primary transceiver. The reflected signal changes based on impedance matching between the antenna 17 and the environment in which the capsule 10 is located, so, given that impedance of the antenna 17 is consistent throughout the passage of the capsule through the GI tract, changes in reflected signal are caused by a change in impedance of the environment in which the capsule is located, and therefore indicate location of capsule within the GI tract. Readings may indicate location by step changes or spikes indicating transition between sections of the GI tract, or by the absolute values being associated with different GI tract sections as determined in a calibration process.

Optionally, in addition to amplitude of reflected signal, the reflectometer may be configured to measure phase of the reflected signal. For example, the capsule 10 may comprise a quadrature demodulator to extract phase information from the reflected signal. Phase information provides a dimension in addition to the amplitude information with which to represent the reflected signal. In a first example, the phase information from the reflected signal may exhibit a step change at a change in environment surrounding the capsule so that analysis of the phase information provides a motility event indicator, which may trigger actuation of the release mechanism. In a second example, discussed below in more detail, the phase information enables a determination to be made of how to modify an antenna control signal to better match the antenna impedance to the impedance of the environment.

Quadrature demodulation converts modulation of the reflectance signal into imaginary and real baseband signals. The quadrature demodulators are driven by carrier frequency (carrier frequency being frequency of transmission by primary transceiver) sinusoids with a 90 degree phase difference to generate two baseband signals that can be compared to generate phase information. Low pass filtering may be applied (to each of the imaginary and the baseband signals) to filter out high frequency content at around double the original baseband frequency.

The reflectometer readings (being one or both of the amplitude and phase readings) provide a basis for differentiating between gaseous, liquid, and solid matter at the location of the capsule in the GI tract, and for differentiating between different sections of the GI tract, so that release timing may be determined. The reflectometer readings (being one or both of the amplitude and phase readings) provide a basis for differentiating between different physical environments surrounding the capsule 10. Readings of the reflectometer enable the antenna 17 and directional coupler 171 to operate in cooperation as an environmental dielectric and impedance sensor.

In a particular example, capsule 10 may be configured to release therapeutic payload directly into the large intestine of the subject mammal 40. The on-board processor may take readings from the reflectometer and process the readings to identify a step change or spike which is identifiable as an indicator of a transition from stomach to small intestine (gastric duodenal transition), and a step change or spike which is identifiable as an indicator of transition from small intestine to large intestine (ileocecal junction transition). Alternatively the readings themselves and in particular value ranges of the readings may be associated with lookup table values to indicate a section of the GI tract in which the capsule 10 is located.

### Reflectometer: Tuneable Antenna

FIG. 3D illustrates a particular example of a reflectometer. Any embodiment having a reflectometer may have a reflectometer as illustrated in FIG. 3D. The capsule 10 is configured to transmit signals from the antenna 17, signals being either data transmission payload or pilot or other signal containing no data and configured to enable reflectometer readings to be obtained.

Since available energy is limited within the capsule 10, the capsule 10 may be configured to transmit signals in an energy-efficient manner. The constrained volume and shape of the capsule 10 in combination with the varying electromagnetic properties of the surrounding environment during transit through the GI tract of the subject mammal mean that impedance matching between the antenna 17 and the surrounding environment is difficult to achieve. Transmission efficiency is improved with better impedance matching. The transmitter 18 may be, for example, control circuitry of the transceiver including a buffer buffering data for transmission.

The transceiver illustrated in FIG. 3B comprises a tuneable antenna 17. A reflected signal from the antenna 17 is generated during transmission, received at the directional coupler 171, and processed at the controller 181 to extract one or both of amplitude and phase information from the reflected signal.

Amplitude provides a measure of amount of reflected energy. Phase information provides information about how the phase shifts between the transmitted and reflected signals. Step changes in either or both may be caused by a change in electromagnetic properties of a transmission environment, that is, an environment in which the capsule 10 is located. Therefore, the reflectometer measurements (being a collective term applied to either or both of the amplitude information and the phase information), either by virtue of their absolute values (and via reference to calibration information such as a lookup table), and/or by virtue of the presence of a step change in their values (in which case calibration information is not required), provide an indicator of an environment surrounding the capsule or of a change in environment surrounding the capsule.

The antenna 17, directional coupler 171, controller 181, variable capacitor 172, form a closed loop mechanism to measure the efficiency of the antenna (wherein amplitude of reflected signal measures efficiency, low amplitude indicating efficiency and high amplitude indicating inefficiency), and to generate a control signal by the controller 181 to a variable capacitor 172 to minimise the antenna reflectance. Depending on how the reflectometer is configured, it may be that the controller 181 is configured to incrementally change the control signal to the variable capacitor 172, to compare amplitude reading with an amplitude reading before the incremental change, and based on the comparison, to determine whether to reverse the direction of the incremental changes or not. Otherwise, in reflectometers that extract the phase information, the phase information itself may inform the controller 181 in which direction the control signal should be varied to reduce the amplitude readings.

The controller 181 is configured, based on the antenna reflectance related readings, to generate a control signal to vary the capacitance of the variable capacitor 172 which varies the impedance of the antenna 17. A control algorithm is responsible for determining the control signal output by the controller 181 to vary the capacitance of the variable capacitor 172 to vary the impedance of the antenna 17 to reduce amplitude of reflected signal from the antenna 17. The controller 181, may generate the control signal empirically by periodically adjusting the control signal in a given direction, comparing a reflectometer amplitude reading pre- and post-adjustment, and changing the direction of adjustment for the next periodical adjustment if the reflectometer amplitude reading increased from pre- to post-, and maintaining the direction of adjustment for the next periodical adjustment if the antenna reflectance related readings decreased from pre- to post. The controller may generate the control signal determinatively based on the reflectometer phase information wherein a particular phase reading value range indicates the controller is to increase the control signal and a particular phase reading value range indicates the controller is to decrease the control signal, and optionally a particular phase reading value range indicates the controller is to maintain the control signal. The level of the control signal generated by the controller 181 is proportional or directly proportional to the capacitance of the variable capacitor 172 and therefore to the impedance of the antenna 17. Since, as detailed above, the antenna 17, controller 181, and variable capacitor 172 form a closed loop or feedback loop mechanism to impedance match (i.e. to reduce reflected signal amplitude) the antenna 17 to the surrounding environment, it follows logically that the control signal generated by the controller 181 to set the capacitance of the variable capacitor is proportional to the impedance of the environment surrounding the capsule 10. Therefore, the control signal may itself be recorded as an antenna reflectance related reading representative or indicative of the environment surrounding the capsule 10.

The reflectometer, configured with the directional coupler 171, controller 181, variable capacitor 172, and antenna 17, forming a closed loop (that is, a feedback loop), provides automatic tuning of the antenna 17 to increase transmission efficiency. Furthermore, the control signal from the controller 181 to the variable capacitor 172, as detailed above, is indicative of impedance of the antenna 17 and therefore also of the environment surrounding the capsule 10, and therefore the control signal may itself be sampled as an antenna reflectance related readings for use in determining release timing. Changes in the control signal or even absolute values of the control signal itself (combined with a calibrated lookup table) provide indicators of capsule 10 location within the GI tract of the subject mammal.

Capsule 10 may be configured to determine release timing when an indicator or plurality of indicators indicate presence of the capsule 10 in a particular section of the GI tract such as the small intestine or large intestine.

The transmitter 18 in the context of FIG. 3D is the circuitry providing the transmission signal (that is, the carrier wave optionally with the encoded data transmission payload, and any metadata etc required by the transmission protocol). The transmitter 18 may be a Bluetooth transmitter.

The readings of the ingestible capsule 10, which include one or more from among readings from: the environmental sensor 14, the heater side 132b of the VOC gas sensor 132, the sensor side 132a of the VOC gas sensor 132, and the TCD gas sensor 131, may also include readings of the reflectometer. Hence, change in capsule location within the GI tract causes a change in antenna reflectance related readings, and therefore provide an indicator that a transition event between two sections of the GI tract has occurred. Reflectometer readings may be used in combination with or instead of gas sensor readings. That is, capsules 10 equipped with reflectometer and configured to take reflectometer readings and to process reflectometer readings to determine release timing do not require gas sensors, and thus gas sensors are optional. However, it is noted that gas sensor readings and reflectometer readings may be combined to obtain indicators of GI tract location. Combining indicators from different sensor type may give a higher degree of confidence than readings from any single sensor type.

### Ingestible Capsule: Accelerometer

As illustrated in FIG. 3E, the ingestible capsule 10 may further comprise an accelerometer 19. The accelerometer 19 is an example of a non-contact sensing mechanism. The accelerometer 19 may be a tri-axial accelerometer. A rate of change of angular position or orientation of the capsule 10 is somewhat dependent upon location within the GI tract, and therefore accelerometer readings provide an indicator that a transition event between two sections of the GI tract has occurred. The accelerometer readings may measure angular acceleration about three axes of rotation, wherein the three axes of rotation may be mutually orthogonal.

Accelerometer readings may be used in combination with or instead of gas sensor readings, or accelerometer readings may be used in combination with reflectometer readings. That is, capsules 10 equipped with accelerometer 19 and configured to take accelerometer readings and to process accelerometer readings to determine release timing do not require gas sensors, and thus gas sensors are optional. However, it is noted that gas sensor readings and accelerometer readings, or accelerometer and reflectometer readings, may be combined to obtain indicators of GI tract location. Combining indicators from different sensor type may give a higher degree of confidence than readings from any single sensor type.

### Accelerometer and Processing of Accelerometer Data

An exemplary accelerometer 19 measures roll about three mutually orthogonal axes. The readings from the accelerometer 19 may be vectors with a component per axis, with each component indicating an instantaneous angular acceleration about the corresponding axis, or an average acceleration about the corresponding axis over the time period since the preceding live reading. Alternatively, the readings may give a three dimensional orientation of the capsule. At the on-board processor 151, at the receiver apparatus 30 or at the remote computer 20, processing of the readings from the accelerometer may be performed to generate a representation (such as a plot vs time) of aggregated (i.e. all three axes) accelerometer readings from which a marker (i.e. an ileocecal junction transition indicator) is identifiable. Such a plot or representation may also be used to identify indicators for motility events and thus for determining release timing, and/or for determining occurrence of an excretion event. In FIG. 9C, a “normalized pitch angle” plot is generated. It is a metric representing cumulative angular displacements of the capsule over time, as measured by the triaxial accelerometer.

A first technique for processing accelerometer data may be referred to as angle travelled (see FIG. 9A). Angle travelled uses vector mathematics to calculate the angle between the gravity vector and a temporary vector. The temporary vector is pulled in the direction of the change in angle, only when this angle exceeds a given threshold (currently 90 Deg). It is then the accumulation of the change in the temporary vector that is visualized in the representation from which markers are identifiable. What is generally seen is that this measure does not change much in the stomach since the angle between the gravity and temporary vectors rarely exceed the threshold in any one direction, (small back and forth orientation changes in the stomach are effectively ignored by the inherent hysteresis of this algorithm) and that once in the tortuous lumen of the small intestine, this measure accumulates significantly due to the larger, more continuous orientation changes of the capsule. Thus, a step change in the cumulative angle travelled measure is a gastric-duodenal transition indicator.

In an exemplary implementation of angle travelled: the accelerometer readings may provide a reading of an orientation of the ingestible capsule relative to a frame of reference in fixed relation to a gravitational vector. Processing of the readings from the accelerometer may comprise recording an orientation of the ingestible capsule given by a first accelerometer reading as a reference orientation, and repetitively in respect of each successive accelerometer reading chronologically: determining whether the orientation of the ingestible capsule given by the respective accelerometer reading is more than a threshold angular displacement from the reference orientation, and if the threshold angular displacement is not met, progressing to the next accelerometer reading without changing the reference orientation, and if the threshold angular displacement is met, changing the reference orientation to align with the orientation of the ingestible capsule given by the respective accelerometer reading. An indicator, such as the gastric-duodenal transition indicator, may be a step change in the rate of change of the reference orientation.

FIG. 9A indicates that a step change in a plot of angle travelled is identifiable within a threshold time period of the detected spike in the TCD gas sensor readings. Therefore, the step change in the plot of angle travelled increases confidence in the hypothesis that the detected spike in the TCD gas sensor readings is caused by gastric-duodenal transition. There are two approximately contemporaneous gastric-duodenal transition indicators, which enables the timing of one of the indicators (which one may be pre-selected, for example, the TCD gas sensor readings) to be determined as the timing of the transition event.

A second technique for processing accelerometer data may be referred to as total roll. Total roll calculates the angle between the gravity vector and each of the capsule X, Y and Z axes and expresses this as a continuous measure that can accumulate beyond 360 Deg. For example, if the capsule x axis is at an angle of 350 Deg and rotates by a further 20 Deg, the resulting angle is expressed as 370 Deg rather than 10 Deg. This helps when representing the readings as a plot from which markers are identified since it avoids the sudden angle changes associated with crossing the zero line. In the example a real change of 20 Deg would be visualized instead of an artificial change of 340 Deg. In addition to this basic approach, low pass filtering may be applied to filter the raw data to remove sensor noise. Additionally, angles are only calculated when the raw accelerometer data provide sufficient data to calculate a meaningful angle. An example of where this is not the case is when the two accelerometer axis values used to calculate the orientation angle around the third axis both approach zero. In this case the calculation will be dominated by sensor noise and so a meaningful angle cannot be determined.

The accelerometer readings provide a reading of an orientation of the ingestible capsule relative to a frame of reference in fixed relation to a gravitational vector. Exemplary processing of the readings from the accelerometer may comprise for each of three orthogonal axes in fixed spatial relation to the ingestible capsule derivable from the reading of the orientation, repetitively in respect of each successive accelerometer reading chronologically: calculating, as a scalar value, a change in the orthogonal axis relative to the gravitational vector from the preceding accelerometer reading; applying a low pass filter to the calculated changes; recording the cumulative filtered calculated changes. A marker serving as a gastric-duodenal transition indicator or ileocecal junction transition indicator may be, for example, an increase (such as a spike or step change) in the rate of increase in the cumulative filtered calculated changes.

### Plots of Sensor Readings for Trial Capsules: FIGS. 9A to 9C

FIG. 9A illustrates a step change in angle travelled at around the same time as a sharp decline in value of corrected TCD readings. It is known that the sharp decline in corrected (i.e. corrected for variation in environmental temperatures) is caused by transition of the capsule 10 across the ileocecal junction, and therefore FIG. 9A is evidence that an indicator of ileocecal junction transition is present in accelerometer readings.

FIG. 9A also illustrates that a gastric duodenal transition (gastric emptying) indicator is present in the corrected (corrected for environmental temperature fluctuations) TCD gas sensor readings (in the form of a spike) and there is a contemporaneous change in the angle travelled plot (though owing to the scale of FIG. 9A the change appears small on the graph. It is further noted that the reflectometer readings, not shown on FIG. 9A, exhibit changes at the gastric-duodenal transition timing and the ileocecal junction transition timing, which changes are indicators of the corresponding motility event. FIG. 9C shows the reflectometer readings and the motility event indicators.

FIG. 9A shows roll in each of three mutually orthogonal dimensions and is marked with gastric emptying event, from which it can be seen that the change in accelerometer readings correlates temporally with the spike in corrected TCD readings (i.e. can be used to add confidence to a detection of gastric-duodenal transition indicator in the temperature corrected TCD readings). FIG. 9A illustrates that the raw readings themselves from each axis of a triaxial accelerometer do contain indicators of both gastric-duodenal transition and ilecocecal junction transition, though the angle travelled metric which combines readings from all three axes shows a clearer indicator. FIG. 9A is further marked with timing of the ileocecal junction transition event from which it can be seen that a step change in angle travelled is detectable and thus provides an indicator of ileocecal junction transition having occurred. The capsule orientation is measured using a triaxial accelerometer and tracking the gravity vector with respect the capsule frame of reference. When the capsule leaves the stomach it tends to experience rapid changes in its orientation as it transits through the duodenum and small intestine. “Angle Travelled”, simply accumulates the orientation change in excess of a 90 degree hysteresis angle. This algorithm tends to be robust to small changes in orientation experienced in the stomach and avoids some of the complexities of other approaches. The 90 degree threshold is exemplary, other threshold angles may be used.

An increase in VOC concentration indicated by the sharp decrease in the motility hot plot of FIG. 9C gives ileocecal junction transition timing. In FIG. 9A the VOC gas sensor readings are not shown, but ileocecal junction transition timing is derivable from the sharp increase in TCD gas sensor reading values (and in accelerometer data). FIG. 9B illustrates the VOC gas sensor readings, referenced as Motility (Hot) in the legend. The sharp drop in the VOC gas sensor reading values is an ileocecal junction transition indicator, and actually represents an increase in VOC concentration in crossing from the small to the large intestine (i.e. the y-axis is inverted with respect to VOC concentration). FIG. 9B further illustrates gastric-duodenal transition timing and the associated rise in corrected TCD reading values, which rise is a gastric-duodenal transition indicator. FIG. 9B illustrates raw accelerometer reading values, from which it can be appreciated that there are step changes at timing of the gastric duodenal transition and ileocecal junction transition. The capsule 10 is configured to process the accelerometer readings by combining the three individual axis plots into a combined metric such as angle travelled as illustrated in FIG. 9A or total roll as illustrated in FIG. 9C. Motility event indicators are identified more readily in the combined metrics.

The traces marked as motility (hot) in the capsule plots are the VOC gas sensor readings. As shown in FIG. 9C the VOC gas sensor readings exhibit an increase in value (representing an increase in concentration of VOCs) at a timing which is attributable to the passage of the capsule across the ileocecal junction. In FIG. 9C, the increase in concentration of VOCs is illustrated by a rise in the values of readings from the VOC gas sensor (i.e. the axis insofar as it relates to the VOC gas sensor readings is positive).

FIG. 9A illustrates an ileocecal junction indicator in the form of a step change in angle travelled metric at timing of the ileocecal junction transition, this step change is an example of a motility event indicator, specifically an ileocecal junction transition indicator. FIG. 9C illustrates the same but in a different accelerometer data metric, total roll.

FIG. 9A illustrates that a gastric duodenal transition (gastric emptying wherein GET is gastric emptying timing) indicator is present in the corrected (corrected for environmental temperature fluctuations).

It is noted, generally, that capsules 10 may determine release timing based on ileocecal junction transition timing, in particular in cases in which the therapeutic matter is intended for delivery directly to the large intestine. Nonetheless, the capsule 10 may still be configured to determine gastric duodenal transition timing. In particular, capsule 10 may determine gastric duodenal transition timing in order to set a lower bound on ileocecal junction transition timing. That is, logically if capsule 10 detects an ileocecal junction transition indicator but the gastric duodenal transition has not yet been determined to have occurred, then capsule 10 (specifically on-board processor) may determine that the detected ileocecal junction transition indicator is not caused by an ileocecal junction transition event. For example, on-board processor may be configured to process one or more from among reflectometer readings, gas sensor readings, and accelerometer readings, to determine timing of gastric-duodenal transition. Once gastric-duodenal transition timing is determined, readings taken after the gastric-duodenal transition are processed to detect ileocecal junction transition indicators and to determine ileocecal junction transition timing and thus release timing of the therapeutic matter.

The plots illustrated in FIGS. 9A and 9C are marked with motility events and motility information derivable from the plot and from which release timing may be determined. Furthermore, should plot readings, motility markers, or other information be generated by the capsule 10 operating in collaboration with receiver apparatus 30 and processing apparatus 20, or operating individually, the motility event indicators may be reported away from the capsule 10 and utilised by a clinician or other operative to gain an understanding of gut health and potentially to diagnose health conditions. Understanding key metrics within the transit of the ingestible capsule through the GI tract. Key metrics include: gastric emptying time (GET), small bowel transit time (SBTT), large bowel transit time (LBTT), and whole gut transit time (WGTT). Any one or a combination of plural of these metrics may be informative to a medical practitioner in assessing the health and/or diagnosing illness or conditions in a subject human. These metrics could be monitored with each capsule ingestion. There may be other biomarkers that are related to the condition being treated that could be monitored also. Conceivably the location for dose deployment could be adjusted for each patient based on this diagnostic information or other information known about the patient, for example if they have particularly issues with the distal colon the deployment of the dose could be timed to be some time after ICJ. The system could also learn how the transit times for a particular patient varies to try and achieve delivery of a dose to the distal small intestine for example (this would assume a somewhat consistent transit time through the small bowel for each patient, or allowing for the variation that is seen over time).

FIG. 9C provides a further illustration of the presence of gastric-duodenal transition indicators in the accelerometer readings (step change in normalized pitch angle) and reflectometer readings 91 (referenced as direct coupler in FIG. 9C), the reflectometer readings showing a gradient change from more or less flat (which would be expected while the capsule 10 is within the stomach and so the dielectric characteristics of the environment surrounding the capsule 10 are consistent), to a negative gradient at the timing of the transition. The gradient change or the negative gradient itself may be detected as gastric duodenal transition indicators by the capsule 10.

FIG. 9C provides a further illustration of the presence of ileocecal junction transition indicators in the accelerometer readings (step change in normalized pitch angle) and reflectometer readings 91 (referenced as direct coupler in FIG. 9C), the reflectometer readings showing a gradient change from more or less flat toward the distal end of the small intestine, to increasing at the timing of the ileocecal junction transition. The gradient change or the positive gradient itself in the reflectometer readings may be detected as an ileocecal junction transition indicator by the capsule 10.

Furthermore, FIG. 9C illustrates the presence of a gastric-duodenal transition indicator in the TCD gas sensor readings (shown as hybrid CO2 in FIG. 9C) in the form of a spike. FIG. 9C also illustrates the presence of a gradient change in the VOC gas sensor readings at the ileocecal junction transition, which gradient change is an ileocecal junction transition indicator.

It is noted that capsule 10 may be configured to have one, two, or more than two sensors operable to generate a gastric-duodenal indicator, and the on-board processor be configured to process the readings from those sensors to identify a gastric duodenal indicator, and may require only one indicator, or may require two or more contemporaneous indicators from different sensors. For example if release timing is to be determined based on gastric duodenal transition timing, or based on a determination that gastric-duodenal transition has been detected.

Similarly, the capsule 10 may be configured to have one, two, or more than two sensors operable to generate an ileocecal junction transition indicator, and the on-board processor be configured to process the readings from those sensors to identify an ileocecal junction indicator, and may require only one indicator, or may require two or more contemporaneous indicators from different sensors.

Wherein contemporaneous is within a predefined length of time of one another, such as five minutes, ten minutes, twenty minutes, or thirty minutes, for example. Optionally, the requirement for one or two or more than two indicators may be determined on-the-fly by the on-board processor based on a characteristic of a first detected indicator, for example, a height of a spike, a magnitude of a gradient change, a magnitude of a variance change etc, so that if a first detected indicator has a characteristic meeting a predefined threshold, no further indicators are required and the motility event is determined to have occurred, or if the characteristic does not the predefined threshold, one or more further indicators within the predefined time window are required for the motility event to be determined to have occurred.

### Data Processing

A representation of the output signal of the VOC gas sensor 132 is transmitted to the remote processing apparatus 20 for processing. The remote receiver 30 may be a smartphone connected to the capsule 10 via Bluetooth, or may be a transceiver in direct wireless communication with the (transceiver 18 of the) ingestible capsule 10 over Bluetooth or a frequency such as 433 MHz. For example, the remote transceiver 30 may comprise a memory readable by the data processor 24. The receiver apparatus 30 may provide a data connection, such as a wired connection, a wireless connection, a network connection, or a plug-socket connection, to the data processor 24 (which may be a computer, a server computer, a cloud computing environment, a smartphone, a tablet). In this manner, the capsule 10 need only be configured to establish a data connection with the remote transceiver 30, which may be a dedicated device for receiving and storing the readings (and optionally with a user interface) and for transmitting a notification signal, or may be a multi-function device such as a mobile phone (such as a smart phone) in order that the subject mammal 40 need not remain within a specific range of the data processor 24 during the live phase. The remote transceiver 30 uploads the representation of the output signal of the VOC gas sensor to the data processor 24. The upload may be continuous during a live phase of the capsule 10. Wherein a live phase is while the ingestible capsule 10 and specifically the VOC gas sensor 132 is actively taking readings. Alternatively there may not be a separate data processor and receiver apparatus, but a single apparatus receiving and processing data from the capsule 10. Capsule 10 may comprise an on-board processor which processes signals, readings, etc from the on-board sensors and triggers the release mechanism 20 on the basis of said on-board processing (i.e. in the absence of any off-board processing).

The data processor 24 may be a cloud resource, or may be a standalone computer at a clinician premise at which the subject is a patient, or may be a server (be it cloud-based or otherwise) at a service provider to which the clinician is a subscriber/customer/servicer user.

For the sake of completeness, it is noted that ingestible capsules configured according to the remote processing functional form may still comprise a microcontroller 15 to control on-board functions such as power distribution, sampling of the output signal, responding to the received notification etc. However the distinction between the two functional forms is that in the local processing functional form the microcontroller 15 itself processes the output signal of the VOC gas sensor 132, whereas in the remote processing functional form the microcontroller 15 coordinates transmission of the output signal to a remote processing apparatus, and receipt and response to a notification signal received in return. Noting in the latter case that the microcontroller 15 may even be considered to be an element or component of the transceiver 18.

Furthermore, the reverse is true. That is, an ingestible capsule 10 configured according to the local processing functional form may still include a wireless transceiver 17, for example in order to realise the reflectometer functionality in a non-contact sensing mechanism. For example, depending on the implementation scenario, the ingestible capsule may be combining the therapeutic payload delivery function with a diagnostic function, so that either the VOC gas sensor 132 itself, and/or one or more additional on-board sensors (such as a TCD gas sensor, an accelerometer, a directional coupler, a temperature sensor, a humidity sensor) generate readings which are transmitted by the wireless transceiver 17 to a receiving apparatus for recording and for further processing. Such additional (i.e. diagnostic) functionality is balanced against power consumption and volumetric capacity considerations depending on the implementation. Volumetric capacity is important since it is advantageous in certain scenarios to maximise payload (i.e. therapeutic payload) capacity whilst suppressing overall capsule size. Furthermore, it is observed that the more power-consuming components are included in the capsule, the more energy is required and hence the greater capacity required for the power source.

In addition, ingestible capsules may implement both the local processing functional form and the remote processing functional form. The third functional form is illustrated in FIG. 3C. The third functional form combines local processing and remote processing to process the output signal of the VOC gas sensor 132 and determine release timing. Therefore, a system implementing the third functional form may have the arrangement illustrated in FIG. 4 (noting that the subject mammal 40 is for illustrative purposes and does not form part of said system).

### Identifying Indicators

The term processing apparatus is used to refer generically to the local processing apparatus (i.e. the microcontroller 15) and the remote processing apparatus 22, and optionally the receiver apparatus 30 to the extent that the receiver apparatus 30 processes data received from the capsule 10.

For example, the microcontroller 15 may be configured to determine release timing by processing the output signal of the VOC gas sensor to identify a release indicator such as an ileocecal junction indicator. In parallel, the microcontroller is transmitting a representation of the output signal of the VOC gas sensor via the wireless transceiver 18 to a remote processing apparatus. The microcontroller 15 is configured to identify particular indicators (which may be referred to as markers) in the output signal, and upon identification of a particular indicator (such as an ileocecal junction indicator), to cause the actuator to release the therapeutic payload from the therapeutic payload carrying compartment at the release timing. The microcontroller 15 may be configured to identify indicators with a very high confidence level (i.e. indicators that to a very high confidence level, such as 95% or higher, or 99% or higher) are causally linked to ileocecal junction transition of the ingestible capsule. In parallel, the remote processing apparatus may be processing the representation of the output signal transmitted via the wireless transceiver 18 to identify indicators. The enhanced processing capacity of the remote processing apparatus may mean it is configured to identify indicators which are not identifiable by the microcontroller 15 (i.e. the microcontroller is configured to identify a subset of the indicators that the remote processing apparatus is configured to identify). Therefore, in some instances the remote processing apparatus will identify a indicator that was not identified by the microcontroller 15. The remote processing apparatus causes transmission of a notification signal to the wireless transceiver 18 to notify the ingestible capsule 10 of the identification of said indicator, and the transceiver is configured, upon receipt of the notification signal, to cause the therapeutic payload to be released from the therapeutic payload carrying compartment by the release actuator.

It is noted that where references are made to an indicator being identified in the output signal of the VOC gas sensor (or a representation thereof), the indicator may actually be an aggregate of a plurality of component indicators. For example, there may be a predefined set of component indicators that the processing apparatus is configured to identify, and it is the cumulative effect of identifying, for example, two, three, or more from among the set that represents identification of the indicator. Examples of indicators include baseline shifts, spikes, step changes, gradient changes, wherein thresholds or other conditions are applied to qualify when an indicator is considered to be identified (indicators in this sense incorporating component indicators).

Depending on the implementation, a plurality of component indicators may be considered an identification of the aggregate indicator if they apply in a predefined order, or the order may be irrelevant. It is further noted that the output signal of the VOC gas sensor may comprise plural components, and so it may be that the indicator being identified is considered to be identified if a component indicator is identified in any one, a particular combination, a threshold proportion, or all, component signal(s).

The indicator being identified by the processing apparatus may be an ileocecal junction indicator, which is an indicator that the capsule 10 has transitioned the ileocecal junction at the interface of the small and large intestine. The ileocecal junction indicator in the output signal of the VOC gas sensor 132 is a characteristic such as a spike, step change or an inflection point in the output signal.

A size, extent, height, or other parameter of the characteristic is indicative of confidence, and therefore the parameter may be used to assess confidence in order to decide whether the characteristic is an indicator and thus should determine release timing, or whether a further corroboratory indicator is required, such as may be provided by the heater side 132b of the VOC gas sensor.

The gas environment change between the small and large intestine is significant due to the large intestine's bacterial population occurring in significantly higher prevalence, driving the creation, or increase, in volatiles and a reduction on O2 through fermentation of carbohydrates and proteins by the microbiota.

The VOC gas sensor output 132 from the sensor side 132a is sensitive to many different volatile analytes with the largest response being due to H2, and O2. At the time of transition through the ileocecal valve a large reduction on the output signal of VOC gas sensor sensing side 132a is observed. As the capsule 10 transits the GI tract the environment is increasingly anaerobic as the O2 is consumed by bacteria.

FIG. 5A illustrates indicators of ICJ on plots of VOC sensor side output signal. The indicator is a characteristic of the output signal. The output signal of the sensor side of the VOC gas sensor in FIG. 5B is represented by the plot that shows a distinct decrease in the highlighted (within the ellipse) region. The remaining plots are from optional further on-board sensors including a TCD gas sensor providing a measure of H2 concentration (when appropriately calibrated), and a temperature sensor measuring temperature of the medium surrounding the capsule.

For example, the output signal may be processed by the processing apparatus on a rolling basis with the output signal for the most recent period of duration t being processed at a time.

FIG. 5B illustrates two plots of output signal against time, with the ICJ indicator being highlighted on each plot within an ellipse. The ICJ indicator may be defined as a decrease in VOC gas sensor output signal followed by an increase in VOC gas sensor output signal with no decrease within a predefined time period after the start of the increase. Wherein the VOC gas sensor output signal may be a magnitude of resistance reading from the sensor side 132a of the VOC gas sensor 132.

In order to determine that the first decrease has occurred, a calibration phase may be initiated by ingestion (which may be determined by markers from on-board sensors such as a temperature sensor, or by a user interaction with an application or interface on a remote computing apparatus) for a predefined period such as two hours, during which time the VOC gas sensor operates as if it were in the live phase, but the processing apparatus (whether on-board or remote) is not actively processing VOC gas sensor output signal to identify the ICJ indicator. During the calibration phase, the processing apparatus records the maximum of the output signal from the VOC gas sensor (for example, the output signal may be the resistance reading of the sensor side 132a of the VOC gas sensor 132). The live phase follows the calibration phase. An output signal drop threshold is predefined. The output signal drop threshold may be predefined as a predefined amount, or a predefined proportion of the maximum output signal recorded during the calibration phase. A first criterion is the output signal dropping more than the output signal drop threshold below the recorded maximum (from the calibration phase). During the live phase, the processing apparatus is monitoring the output signal of the VOC gas sensor for a decrease beyond the output signal drop threshold below the recorded maximum. Optionally, the first criterion may be considered satisfied, in the case of the output signal being composed of discrete readings, when n or more consecutive readings are more than the output signal drop threshold below the recorded maximum, wherein n may be 1, 2, 3, 5, or 10. In the case of the output signal being a continuous signal, the first criterion may be considered satisfied when the level of the output signal is more than the output signal drop threshold below the recorded maximum for more than a preset continuous time period, wherein a duration of said time period may be 0.1 second, 0.5 second, 1 second, 2 seconds, three seconds, 5 seconds, or 10 seconds. A second criterion may be that the output signal increases after the first criterion is satisfied without decreasing within an immediately subsequent time window of predefined duration (for example, ten minute, fifteen minutes, twenty minutes, thirty minutes). Decreasing in the context of the second criterion may be a negative gradient (for more than 1, 2, 3, 5, or 10 consecutive readings, or continuously for 0.1 second, 0.5 second, 1 second, 2 seconds, three seconds, 5 seconds, or 10 seconds) or may be dropping below a minimum defined at the start of the increase. The indicator is identified by virtue of the first and second criteria being satisfied. That is, the satisfaction of the first and second criteria is identification of a predefined characteristic in the sensor side output signal. The release actuator is activated (immediately or following a predefined delay) once the indicator is identified.

Alternatively, the indicator may be identified through plotting the differential of the VOC sensor side output signal vs time whilst the sensor is heated and finding a negative peak at or above a threshold size. This differential locates the point of change which is associated with the transition across the ileocecal junction but does not occur at the start of the transition event. The start of the transition event may be found by the initial inflection point from the baseline in the first derivative. However, embodiments do not necessarily require the particular event timing, rather, to determine with a predefined level of confidence that the transition across the ileocecal junction has occurred within a short (i.e. less than a minute) time of its occurrence. The start of the transition event may be a useful piece of information for health and diagnostic purposes, in assessing motility of the capsule 10 through the GI tract.

As illustrated in FIG. 6, an ICJ transition indicator is also present in the determined H2 concentration percentage, as a sharp increase in H2 when the capsule reaches the colon. The H2 produced in the GI tract is a byproduct of fermentation. The colonies of bacteria are orders of magnitude larger in the colon than in the small bowel. Therefore, determined H2 concentration may be used to add confidence to the ileocecal junction indicator in the VOC sensor output. The H2 concentration may be measured by the output signal of the VOC gas sensor heater side 132b, which is indicative of thermal conductivity in the gas surrounding the heater. For example, if the negative peak identified in the differential of the VOC sensor side output signal vs time does not exceed the threshold, but does exceed a second, lower, threshold, then a corroboratory indicator may be sought in the output signal of the VOC gas sensor heater side 132b, which by calibration and/or by further processing at the processing apparatus can be linked to H2 concentration.

Commercial bands (such as 433 MHz) are used by the antenna 17 as electromagnetic waves in this frequency range can safely penetrate the mammalian tissues 40. Other commercial bands may be used in various applications, such as Bluetooth. Coding may be applied at the digitisation stage to assure that the data transmitted by the capsule 10 is distinguishable from data transmitted by other similar capsules 10. The transmission antenna 17 may be, for example, a pseudo patch type for transmitting data to the outside of the body data acquisition system. Power source 16 is a battery that can supply the power for the sensors and electronic circuits. A life time of at least 48 hours is required for digestive tract capsules. A number of silver oxide batteries in the power source 16 is configurable, depending on the needed life time and other specifications for the capsule.

### Power On

Capsule 10 may be powered on by removal from packaging which breaks a seal isolating the power supply from the electronic components, thus initiating a live phase of the capsule, which live phase continues, for example, until the power supply is exhausted or until the therapeutic payload is released.

Power on may be via an NFC handshake between an NFC transceiver on-board the capsule and an NFC transceiver of a smartphone running an application specifically configured to manage the powering on, processing and storing of data received from the capsule 10.

Other power on options include a switch beneath a flexible portion of housing, and receipt of high intensity light with encoded data in light from, for example, a smartphone screen running an application for managing interaction between smartphone 30 and capsule 10.

A reed switch held in a closed position by a magnet in capsule packaging and opened by separation of the capsule from the capsule packaging is a further example of a power on or initiation mechanism.

### Release Actuators: FIG. 7A

FIG. 7A illustrates an ingestible capsule 10 with a biocompatible indigestible housing. In particular, FIG. 7A illustrates an exemplary release actuator 21, including a compressed spring 211, a fuse wire 212, and a puncturing member 213.

The capsule 10 has an aperture 224 at an end, a headspace at the end being defined by a flexible membrane 222 of biocompatible indigestible polymer. The headspace is isolated from the remainder of the capsule interior by the flexible membrane 222. A therapeutic payload carrying compartment 22 is adhered or otherwise attached to the flexible membrane 222. A payload of therapeutic payload 221 is contained within the therapeutic payload carrying compartment 22. A perforable wall of the therapeutic payload carrying compartment 22 faces the aperture 224 and faces a puncturing member 213. Said perforable wall is formed of a biocompatible material that is perforable by the puncturing member 213 upon application of a pushing force. Examples include biocompatible indigestible polymers and biocompatible indigestible foils. A compressed helical spring 211 is pre-coiled and held in a coiled state by a fuse wire 212. The spring 211 abuts a rigid/inflexible surface 225 at an end distal the aperture 224, and abuts the flexible membrane 222 at an end proximal the aperture. At a timing determined by the release mechanism 20, the fuse wire 212 is released, causing the spring to uncoil against the rigid/inflexible surface 225 and thus to push the flexible membrane 222, thus pushing the perforable wall 223 into the puncturing member 213, causing the therapeutic payload 221 to be released into the GI tract via the aperture 224. A shape memory alloy wire could also be used to release a spring loaded mechanism.

For example, the reed switch 23 cooperates with a microcontroller to control power supply to the VOC sensor 132. A gas permeable membrane 12 defines a headspace at an end of the capsule 10 distal the aperture 224. The VOC sensor 132 is housed within the defined headspace, which may be in fluid isolation from the remainder of the capsule interior, but with connectivity between the battery power 16, reed switch 23, microcontroller 15, and VOC sensor 132 for exchange of power and output signal.

### Release Actuators: FIGS. 7B, 7C, 7D, 7E, 7F

FIG. 7B illustrates a further exemplary release actuator. FIG. 7B is to illustrate a particular release actuator arrangement and is not intended to illustrate all capsule components.

In broad terms, the release actuator of FIG. 7B is a heating element 743 mounted on a PCB 740 and configured to rupture an elastic material membrane 722. The therapeutic payload carrying compartment 22 comprises a section of the ingestible capsule housing and a sealed chamber, the elastic material membrane 722 defines at least a portion of a wall of the sealed chamber, within which sealed chamber the therapeutic payload is sealed.

The sealed chamber may be a balloon or may be an elastic material membrane 722 stretched over a rigid open frame component 770, as illustrated in FIG. 7D. The sealed chamber may carry the therapeutic matter itself, or may carry a liquid diluent for mixing with the therapeutic matter (see FIG. 7C).

FIG. 7D illustrates a rigid open frame component 770, and an arrangement of rigid open frame component 770 and elastic material membrane 722 to form a sealed chamber to fit within the therapeutic matter carrying compartment 22 of the ingestible capsule 10.

FIG. 7D is composed of a series of images (i) to (vi) illustrating a sealed chamber being formed, incorporated into a capsule, and unsealed at the determined release timing. In image (i), the rigid open frame component 770 is illustrated. The rigid open frame component 770 is dimensioned to fit within the therapeutic matter carrying compartment 22 of the ingestible capsule 10 and defines an interior volume to carry a dose of the therapeutic matter or liquid diluent, as required. The rigid open frame structure 770 defines a plurality of apertures, which are to be sealed in order to form the sealed chamber.

At image (ii), an elastic material membrane 722 is stretched over the rigid open frame component 770. The elastic material membrane 722 is stretched, that is, exerts a tensional force across the membrane. In other words, the sealed chamber may be defined by a stretched elastic material membrane 722. Bonding may be applied between the elastic material membrane 722 and the rigid open frame component 770 in order to secure the sealing between the two entities.

The elastic material membrane 722 may be formed of an elastomer, which elastomer may be rubber, latex, nitrile, or another elastomer, or a combination of one or more of those.

At image (iii), therapeutic matter or liquid diluent is inserted into an unsealed or open chamber defined by the rigid open frame component 770 and the elastic material membrane 722, via a further aperture (i.e. further aperture is not sealed by the elastic material membrane 722).

At image (iv), sealing of the chamber by closing the further aperture is illustrated. In particular, the further aperture is closed by an outer cover 723. The outer cover 723 may be bonded to the frame at the edge of the further aperture, as illustrated in image (iv). The outer cover may form part of the housing of the ingestible capsule 10 or may be separate from the housing and dimensioned to sit within the housing of the ingestible capsule 10.

At image (v) the sealed chamber or sealed container or sealed pod is illustrated bonded to the other components of the ingestible capsule 10. The elastic material membrane 722 is in contact with a heating element 743 on the printed circuit board 740 defining an inner surface of the therapeutic payload carrying compartment 22. Apertures 760 in the housing of the capsule 10 are closed (i.e. fluid communication between the interior and exterior of the capsule 10 at the apertures 760 is prevented) by the elastic material membrane 722 inside the housing. Heating element 743 may be mounted on printed circuit board 740 or integrally formed with printed circuit board 740.

At image (vi) the arrows illustrate that the heating element 743 has ruptured the elastic material membrane 722 thereby the sealed chamber defined thereby is no longer sealed, and the therapeutic payload is allowed to exit the capsule 10 via the apertures 760. In the particular example of FIG. 7D, the elastic material membrane 722 is in a stretched state forming a part of a wall of the sealed chamber, and therefore upon rupturing the elastic material membrane 722 is pulled apart by a tensioning force, thereby facilitating opening of the sealed chamber and the passage of the therapeutic payload out of the capsule 10 and into the GI tract.

Returning to FIG. 7C, the sealed chamber is filled via the one-way valve 730, which seals an aperture 731. In particular, it is noted that aperture 731 is an aperture in the housing 11 allowing the sealed chamber to be filled after manufacture of the capsule 10. Such an aperture 731 is optional, since, as illustrated in FIG. 7D, sealed chamber may be filled and sealed during manufacture of the capsule 10, in which case no such aperture 731 and valve 730 arrangement is required.

A supercapacitor 716 is trickle charged by the power source 16 once the capsule 10 is powered on. At the determined release timing, microcontroller 15 causes the supercapacitor to discharge into the heating element 743, causing the heating element 743 to increase in temperature at a rate sufficient to rupture a part of the wall of the sealed chamber formed by the elastic material membrane 722. The sealed chamber is caused to unseal, thus releasing the therapeutic matter and, via one or more apertures in the capsule housing (aperture(s) not illustrated) into the GI tract of the subject mammal 40. It is noted that the sealed chamber may be a balloon which is expanded into the therapeutic matter carrying compartment 22 so that an outward pressure is exerted on the wall of the balloon by fluid therein. In the balloon example, the rupturing causes the balloon to burst and the therapeutic payload is forced outward and out of the apertures 760 upon bursting.

The capsule 10 includes further rigid printed circuit boards 741 on which electronic components are mounted. The supercapacitor 716 is connected to the rigid circuit board on which the heating element 743 is mounted 740 via a section of flexible circuit board 742 which provides a low resistance connection between supercapacitor 716 and heating element 743 in order to ensure that energy stored by the supercapacitor is dissipated by the heating element 743 and not by the electrical connection between the supercapacitor and the heating element 743, insofar as that is practicable.

FIGS. 7E & 7F illustrate a sectional view, and an isometric sectional view, of an ingestible capsule 10. FIG. 7E illustrates a gas permeable membrane (specifically permeable by GI tract gases) 12, which membrane is omitted in FIG. 7F. The omission of the membrane in FIG. 7F is for illustrative purposes: capsules 10 either have a gas permeable membrane 12 covering an aperture (in the case of a direct gas sensing mechanism), or a covered end (in the case of a non contact sensing mechanism). Noting that capsule 10 may have both direct gas and non-contact sensing mechanisms, in which case gas permeable membrane 12 is required. Rigid circuit boards 740 provide a substrate on which, for example, wireless transceiver 18, microcontroller 15, and optionally accelerometer 19 and/or other electronic components are mounted. Supercapacitor 716 is trickle charged by power supply 16 and transfers charge to the heating element 743 at the determined release timing. Elastic material membrane 722 is stretched over rigid open frame 770 to form a sealed chamber for carrying the therapeutic payload. At determined release timing, transfer of energy from supercapacitor 716 to heating element 743 causes elastic material membrane 722 to increase in temperature sufficiently to rupture. Tensional force in the elastic material membrane 722 causes the elastic material membrane to pull away from the rigid open frame 770, thereby permitting the fluid from the GI tract to mix with the therapeutic payload via the apertures 760. In particular, FIGS. 7E and 7F illustrate a space defined between the printed circuit board 740 on which the heating element 743 is mounted and the rigid open frame at 771, thereby permitting the heating element 743 to contact the elastic material membrane 722 in a space having reduced or no fluid communication with the rest of the therapeutic payload carrying compartment 22. Thereby, heat dissipation from heating element 743 to surrounding fluid is inhibited and heat dissipation from heating element 743 to elastic material membrane 722 is promoted.

A sealed chamber for carrying the therapeutic payload (therapeutic matter) may be formed by stretching an elastic material membrane 722 over a rigid open frame 770, or by inflating an elastic material membrane 722 defining a balloon.

A balloon is a bag or receptacle having a sealable aperture, via which aperture the balloon is configured to receive content, the balloon being configured to expand into space around the balloon in response to pressure exerted by received content. In particular, content is forced into the balloon at a pressure exceeding pressure of the environment surrounding the balloon, thereby causing the balloon to expand into surrounding space. The aperture is sealable, for example via a one-way valve or some other mechanism, thereby preventing content from leaving the balloon and fixing the balloon in the expanded state, assuming the environmental pressure does not change, or specifically does not increase to beyond the pressure inside the balloon. The balloon with the valve is an example of a sealed chamber formed by an elastic material membrane for containing or carrying the therapeutic payload.

Owing to retaining content at a pressure exceeding environmental pressure, the balloon is prone to bursting upon piercing or rupturing of the bag. Bursting is release of content stored within the balloon into the surrounding environment, the pressure differential prior to the bursting event causing the content to project outwardly upon balloon bursting.

Optionally, the balloon is stretchable, for example comprises a bag formed of an elastomer. The elastomer may be rubber, latex, nitrile, or another elastomer, or a combination of one or more of those.

Alternatively the balloon may be non-stretchable, for example formed of foil.

Capsule 10 may comprise a balloon contained within a section of the housing that is configured to retain the balloon within the housing but which is not sealed to the surrounding environment, so that once the balloon is ruptured the content of the balloon is no longer retained within the capsule and, either by ejection from the balloon bursting, or by mixing with environmental fluids allowed into the housing and then leaving the housing. For example, the housing may comprise one or more apertures 760, windows, or perforations allowing the exchange of fluid and/or solid matter between the interior of the capsule and the surrounding environment.

The section of the housing configured to retain the balloon may be separate from a sealed section of the housing configured to retain the electronic components of the capsule 10.

In the case of the balloon within the capsule 10, the content comprises therapeutic matter and fluid, wherein the fluid may be a gas such as air, and/or liquid such as a liquid diluent.

As illustrated in FIG. 7D, the capsule 10 may comprise a rubber encapsulated open frame that can be filled and assembled into the capsule 10, wherein rupturing of the rubber would cause the rubber to peel away from the frame and release the contents into the surrounding environment.

A sealed chamber at least partially formed by the elastic material membrane 722 or formed by a balloon may be filled at manufacture and provided to clinicians loaded with the therapeutic matter, and sealed. Alternatively the sealed chamber may be filled at clinician or pharmacy via a mechanism such as a syringe or equivalent configured to insert content into the balloon via a one-way valve at the balloon aperture.

The valve may be, for example, an umbrella valve, a Belleville valve, a ball valve, or a dome valve, exemplary of one-way valves that allow for the sealed chamber to be filled with fluid while forming a seal between the interior of the balloon and the environment external to the balloon.

As an alternative to the valve, the sealed chamber may comprise a septum seal into which balloon filling content is injectable with a needle of a syringe or equivalent. In this example, the balloon material at the septum seal forms a seal as the filling needle is retracted.

As a further alternative to the valve, the filling of the sealed chamber may be via an extruded tube protruding out from the ingestible capsule 10 via an aperture and which extruded tube, after filling of the chamber, is subsequently heat staked down to both seal the chamber and to provide a smooth exterior shape for ingestion. Optionally, a one-way valve may be included beyond the tube to hold back pressurised fluid during a period when the chamber is filled with therapeutic payload but the tube has not yet been heat staked. A further variation would be filling the chamber through an aperture and then sealing the chamber by welding on a lid or cover over the aperture.

In FIG. 7B, the therapeutic matter is contained within a sealed chamber, part of a wall of which sealed chamber is formed by an elastic material membrane 722. The sealed chamber is fillable with content including the therapeutic payload. The elastic material membrane 722 may be stretched over a rigid open frame, or may be a balloon, the therapeutic payload carrying compartment being arranged to allow the balloon to expand to fill the therapeutic carrying compartment 22 of the ingestible capsule 10 when the balloon is filled.

The release actuator comprises an elastic material membrane rupturing mechanism configured, at the determined release timing, to rupture the elastic material membrane 722 thereby allowing the content of the sealed chamber defined by the elastic material membrane 722 and including the therapeutic payload to exit the ingestible capsule via one or more apertures in the section of the ingestible capsule housing containing the sealed chamber. In FIG. 7B, the elastic material membrane rupturing mechanism is a heating element 743 such as a wire and a power source 716 configured to provide power to the heating element 743 to cause the heating element 743 to heat up at a rate sufficient to cause the elastic material membrane 722 to rupture.

The heating element 740 is arranged at least partially within, or against an interior surface of, the therapeutic payload carrying compartment 22, the elastic material membrane 722 being arranged to expand to fill the therapeutic carrying compartment and thereby to contact the heating element 743. An internal wall at least partially formed by printed circuit board 740 may define a surface or wall of the therapeutic matter carrying compartment 22.

The power source 716 of the balloon bursting mechanism may be a battery or may be a supercapacitor configured to be trickle charged by the ingestible capsule power supply following an initiation event of the ingestible capsule, and to be caused to release the charge to the heating element 743 at the release timing under the control of the microcontroller. The battery or supercapacitor 716 may be impedance matched to the heating element 743 to improve power transfer to the heating element and optimise the rate of heating of the elastic material membrane 722. Impedance matched is taken in this context to mean matched to within a defined tolerance. The defined tolerance may be, for example, 1% or less, 5% or less, 10% or less, 15% or less, 20% or less, 25% or less. Impedance matching may include the connector between the supercapacitor 716 and the heating element 743 as part of the heating element 743.

In order for the heating element 743 to rupture the elastic material membrane 722, it must receive energy at a rate sufficient to exceed energy loss via thermal transfer away from the heating element 743, noting that said energy loss increases as heat differential between heating element 743 and surroundings increases. To improve power transfer between the power source and the heating element 743, the supercapacitor and the heating element 743 may be impedance matched.

Optionally, a rigid open frame about which the elastic material membrane 722 is stretched, and/or an internal wall of the capsule 10, and or the printed circuit board 740 about the heating element 743, is configured to inhibit fluid flow about the heating element 743, in order to reduce heat loss to surroundings and to promote heat dissipation to the elastic material membrane 722.

Optionally, the therapeutic matter carrying chamber may comprise a deployment detecting mechanism for detecting that the therapeutic payload has been deployed into the GI tract. For example, a pair of electrodes at a surface of the compartment covered by the elastic material membrane 722 while the sealed chamber is in a sealed state, and uncovered when the elastic material membrane 722 is ruptured so that impedance of the electrodes changes and release is positively detected. Electrodes may be connected to a microcontroller so that information representing positive detection of therapeutic payload release and a timing thereof may be included in a report to be transmitted away from the capsule 10, for example via the wireless transceiver 18.

The report may be transmitted immediately after detection, or once capsule excretion is detected, or at some other timing. Alternatively or additionally, the report may include the determined release timing and optionally also information representing the ileocecal junction transition indicator(s) or gastric-duodenal transition indicator(s) based upon which release timing was determined.

The deployment detecting mechanism may comprise conductive pads on a printed circuit board assembly (i.e. providing one or more from among processor hardware, memory hardware, wireless transceiver and other reflectometer components, microcontroller, power management, etc) next to the heating element 743, the impedance across the conductive pads being measured periodically by the microcontroller or equivalent, to identify a change in impedance when the elastic material membrane 722 transitions from a stretched state (in which it lies across the conductive pads and forms part of the wall of the sealed chamber) to a ruptured state in which fluids from the GI tract and/or sealed chamber are present in the capsule interior at the location of the conductive pads.

Alternative deployment detecting mechanisms include optical presence detection, capacitive sensing, or a mechanical switch.

In an alternative to the arrangement illustrated in FIG. 7B, the elastic material membrane rupturing mechanism may comprise a power source, a shape memory alloy wire, and a rupturing member, the power source being configured, at the determined release timing and under control of the microcontroller, to transfer energy to the shape memory alloy wire, to initiate a phase change at material level of the shape memory alloy wire and thereby to exert a force on the rupturing member to cause the rupturing member to come into contact with, and to rupture, the elastic material membrane 722. For example, shape memory alloy wires undergo a shortening of 5-10% at the phase change, which shortening exerts a pulling force that can directly or indirectly cause the elastic material membrane 722 to rupture.

As a further alternative, the elastic material membrane rupturing mechanism may comprise a motor and a rupturing member, the microcontroller being configured, at the determined release timing, to power on the motor and thereby to exert a force on the rupturing member to cause the rupturing member to come into contact with, and to rupture, the elastic material membrane 722.

FIG. 7C illustrates an alternative to the therapeutic payload (therapeutic matter) being contained within the sealed chamber. In the capsule 10 of FIG. 7C, the therapeutic matter 750 is within the therapeutic matter carrying chamber 22 but not within the sealed chamber. Specifically, the therapeutic matter 750 is in powdered, dehydrated, or otherwise solid form and is arranged within the therapeutic matter carrying chamber 22 so that it is sealed from the environment external to the capsule 10 by the elastic material membrane 722. Noting in particular that there is at least one aperture 760 allowing exchange of fluids between the therapeutic matter carrying compartment 22 of the capsule 10 and the external environment (also present in FIG. 7B though not illustrated therein), and that the elastic material membrane 722 prevents communication of fluid between the external environment and the portion of the therapeutic matter carrying chamber 22 housing the therapeutic matter 750.

In the example of FIG. 7C the sealed chamber is filled with liquid diluent, so that when the elastic material membrane 722 is ruptured, the liquid diluent is mixed with the therapeutic matter and the mixture is allowed to mix with the fluid from the environment external to the capsule 10 via the aperture or apertures 760, noting that the apertures are dimensioned to enable sufficient time for the liquid diluent to mix with the therapeutic matter before exiting the capsule 10.

The heating element 743 may be a resistive heater element comprising one or more from among: SMT resistor; metallic resistive wire; nichrome; MEMS hearer element.

In an alternative balloon bursting mechanism, the mechanism comprises a power source and a LASER diode focussed on the elastic material membrane 722, wherein a microcontroller of the ingestible capsule is configured at the determined release timing to activate the LASER diode to rupture the elastic material membrane 722.

In a further alternative, the rupturing mechanism comprises a pre-sprung mechanical needle, wherein a microcontroller of the ingestible capsule is configured, at the release timing, to release the pre-sprung mechanical needle causing the pre-sprung mechanical needle to spring into the elastic material membrane 722 and cause the elastic material membrane 722 to rupture.

In a further alternative, the release mechanism comprises a microcontroller and a release actuator, and the therapeutic payload carrying compartment comprises a portion of the ingestible capsule housing containing a balloon, the balloon being fillable with balloon content including the therapeutic payload, the therapeutic payload carrying compartment being arranged to allow the balloon to expand to fill the therapeutic carrying compartment when the balloon is filled. The balloon comprises a releasable valve; the release actuator comprising a releasable valve activating mechanism configured, at the determined release timing, to activate the releasable valve thereby allowing the balloon content including the therapeutic payload to exit the ingestible capsule via one or more apertures in the portion of the ingestible capsule housing containing the balloon.

For example, the releasable valve may be a kinked hose, wherein the releasable valve releasing mechanism is a shape memory alloy wire or micro motor configured, under control of a microcontroller of the ingestible capsule, to unkink the hose thereby unsealing the chamber and allowing content of the chamber to exit the ingestible capsule via one or more apertures in the housing of the ingestible capsule.

In a further alternative balloon bursting mechanism, the elastic material membrane rupturing mechanism comprises a power source, a shape memory alloy wire, and a rupturing member, the power source being configured, at the determined release timing and under control of the microcontroller, to transfer energy to the shape memory alloy wire, to initiate a phase change at material level of the shape memory alloy wire and thereby to exert a force on the rupturing member to cause the rupturing member to come into contact with, and to rupture, the elastic material membrane, thereby unsealing the sealed chamber.

### Identifying Indicators and Sensor Arrangements

In addition to (i.e. as additional sensor outputs in which ICJ indicators are identifiable to add confidence to the indicator identified VOC gas sensor output) or as alternatives to the VOC gas sensor output (i.e. in embodiments which either do not have a VOC gas sensor, in which the VOC gas sensor is faulty, or in which the indicator is for some reason not identifiable in the VOC gas sensor output signal), one or a combination of the following sensor outputs and indicators may be processed and identified:
TCD gas sensor (more information provided below), from which a plot of H2 concentration vs time is derivable by appropriate calibration, the ICJ indicator being a steep rise (i.e. positive gradient above a predefined threshold) in H2 concentration;
TCD gas sensor, from which a plot of CO2 concentration vs time is derivable by appropriate calibration, the ICJ indicator being a steep rise (i.e. positive gradient above a predefined threshold) in CO2 concentration. FIG. 8 illustrates an ICJ indicator in a plot of CO2 concentration vs time;
Reflectometer formed by the antenna 17 and directional coupler 171 (FIG. 3B, FIG. 3D, FIG. 3E), the ICJ indicator being a step change or spike in the reflectometer output (see below);
Accelerometer (see FIG. 3E).

Optionally, the ingestible capsule further comprises a directional coupler in series with the antenna to form a reflectometer, and the output signal of the reflectometer is processed at the remote processing apparatus to identify an ICJ indicator therein. The antenna 17 may be in series with a directional coupler 171. The directional coupler 171 and the antenna 17 are configured as a reflectometer. The reflectometer measures the amplitude of reflected signals by means of a diode detector. The measurements of the reflectometer are readings that represent electromagnetic properties of material in the vicinity of the capsule. The reflectometer readings provide a basis for differentiating between gaseous, liquid, and solid matter at the location of the capsule in the GI tract. Readings of the reflectometer enable the antenna 17 and directional coupler 171 to operate in cooperation as an environmental dielectric sensor.

The capsule may generate an output signal from readings of the reflectometer for transmission via the capsule transceiver to a remote processing apparatus for identification of an ICJ indicator. Hence, change in capsule location within the GI tract causes a change in reflectometer readings, and therefore provide an indicator that a transition event between two sections of the GI tract has occurred.

The capsule may include a TCD gas sensor disposed alongside the VOC gas sensor in the headspace at an end of the capsule sealed from the external environment by a gas-permeable membrane and sealed from the remainder of the capsule interior. Embodiments also include the possibility of TCD gas sensor functionality being provided by the output of the heater side of the VOC gas sensor.

The heater side of the VOC sensor (operating as a TCD sensor) and the sensor side of the TCD sensor have different operating ranges, so TCD readings from the two sensors collectively span a wider range of operating temperatures than either of the sensors individually. Both sensors have heating elements. The TCD sensor has a low operating temperature but with a high precision. The heater side of the VOC increases the operating range but has a lower precision for TCD readings than the TCD sensor. The larger collective thermal range achieved by the two gas sensors in concert enables better resolution of analytes in the processing of the output signal(s). The thermal conductivity of constituent gases in the gas mixture of the GI tract varies with temperature and so by obtaining TCD readings at different operating temperatures the different gases can be resolved from each other. This is leveraged in processing the output signal to identify ICJ indicators, which is via determining identity and concentrations of constituent gases in the gas mixture surrounding the capsule 10.

The gas sensors are contained in a portion of the capsule 10 sealed from the power source 16 and other electronic components. At least a portion of the outer surface of this portion of the capsule is composed of a selectively permeable membrane. For example, the gas sensors include respective heaters which are driven to heat sensing portions of the respective gas sensors to temperatures at which sensor readings are obtained (i.e. a measurement temperature). The heaters may be driven in pulses so that there is temporal variation in the sensing portion temperature and so that measurement temperatures are obtained for periods sufficient to take readings but without consuming the power that would be required to sustain the measurement temperature continuously.

The gas sensors may be calibrated, so that a gas sensor reading can be used to identify the composition of the gas mixture of the environment in which the capsule is located and optionally also concentration of a particular gas (i.e. particular component(s) of the gas mixture). Capsule 10 may also be configured to operate in the absence of such calibration, for example where changes in gas sensor signals are utilised to identify motility events such as gastric duodenal transition or ileocecal junction transition (further noting that capsules may be configured without gas sensors). Calibration coefficients are gathered in manufacturing and applied to the recorded readings at the processing stage (i.e. by a remote processing apparatus). Otherwise, this calibration could be performed on the capsule 10, at the remote processing apparatus, or on any device having access to the calibration coefficients and the recorded readings from the gas sensors. Such calibration relates to processing concerned with measuring the concentration of constituent gases in the gas mixture at the capsule, in order to identify ICJ indicators as additional to, or alternative to, the ICJ indicator in the VOC gas sensor side output signal. TCD readings are effectively measuring rate of heat loss to surroundings, and so accuracy is improved by measuring the temperature of the surroundings rather than by relying on assumption (i.e. prior knowledge of internal temperature of the subject mammal). However, the processing may rely on assumption, for example, if the capsule 10 does not include an environmental temperature sensor or if there is some issue with the environmental temperature sensor readings, or, for example, if the level of accuracy provided by assumption is acceptable in a particular implementation. Environmental temperature is a term used in this document to refer to the temperature of the environment in which the capsule 10 is located, as distinct from operational temperatures of the gas sensors. The sensitivity of the gas sensors 13 to different constituent gases vary according to the operating temperature of the sensors and the processing of the readings includes calibrating (also referred to as moderating or correcting) readings from the gas sensors according to contemporaneous operating temperature and optionally also according to contemporaneous environmental temperature.

FIG. 10 is a schematic illustration of a hardware arrangement of a remote processing apparatus. The remote processing apparatus 24 may be implemented by one or more apparatus having an arrangement such as illustrated in FIG. 10.

The remote processing apparatus 24 comprises a plurality of components interconnected by a bus connection. The bus connection is an exemplary form of data and/or power connection. Direct connections between components for transfer of power and/or data may be provided in addition or as alternative to the bus connection.

The remote processing apparatus 24 comprises memory hardware 991 and processing hardware 993, which components are essential regardless of implementation. Further components are context-dependent, including a network interface 995, input devices 997, and a display unit 999.

The memory hardware 991 stores processing instructions for execution by the processing hardware 993. The memory hardware 991 may include volatile and/or non-volatile memory. The memory hardware 991 may store data pending processing by the processing hardware 993 and may store data resulting from processing by the processing hardware 993.

The processing hardware 993 comprises one or a plurality of interconnected and cooperative CPUs for processing data according to processing instructions stored by the memory hardware 991.

Systems may comprise one remote processing apparatus according to the hardware arrangement of FIG. 10, or a plurality of such devices operating in cooperation with one another.

A network interface 995 provides an interface for transmitting and receiving data over a network. Connectivity to one or more networks is provided. For example, a local area network and/or the internet. Connectivity may be wired and/or wireless.

Input devices 997 provide a mechanism to receive inputs from a user. For example, such devices may include one or more from among a mouse, a touchpad, a keyboard, an eye-gaze system, and a touch interface of a touchscreen. Inputs may be received over a network connection. For example, in the case of server computers, a user may connect to the server over a connection to another computing apparatus and provide inputs to the server using the input devices of the another computing apparatus.

A display unit 999 provides a mechanism to display data visually to a user. The display unit 999 may display user interfaces by which certain locations of the display unit become functional as buttons or other means allowing for interaction with data via an input mechanism such as a mouse. A server may connect to a display unit 999 over a network.

The network interface may comprise, or be in communication with, a remote processing apparatus transceiver in data communication with the capsule 10.

### Ulcerative Colitis (UC)

Therapeutic matter carried by the therapeutic matter carrying compartment and released in to the GI tract by the UC may be a pharmaceutical formulation for the treatment of UC. In particular the pharmaceutical formulation may be a corticosteroid such as budesonide, or a 5-aminosalicylate such as pharmaceutical. Embodiments enable pharmaceutical formulations to be released with precision into the colon so that dosage has high efficacy compared with conventional methods for administering pharmaceutical formulations intended to treat a condition of the colon. Furthermore, embodiments are configurable to either release a dose as a single discrete dose at a release timing that may be, for example, immediately following detection of ICJ transition, or as a series of partial doses at a series of partial release timings separated by a predefined interval. A capsule 10 may be configurable at manufacture or post-manufacture by a clinician (for example causing a signal to be received by the microcontroller to change configuration). In particular, inflamed tissue associated with UC may be all in the first part of the colon (best treated by single dose), all at the distal end of the colon (best treated by series of partial doses), or distributed all along the colon (best treated by series of partial doses). Clinician may be able to determine the distribution of inflamed tissue associated with UC in the subject and thus to select single discrete dose or series of partial doses. Said selection may comprise selecting an appropriate pre-loaded capsule, or may comprise configuring the microcontroller 15 of a capsule 10 to behave in a certain way by sending a signal from a controller apparatus that is received at the microcontroller 15. There are toxicity effects associated with consumption of pharmaceutical formulations, which are balanced against efficacy. Embodiments may improve the balance somewhat in favour of efficacy by delivering therapeutic matter to the site of the inflammation associated with UC and in a personalised manner (i.e. in a single discrete dose or series of doses at intervals). UC is used as an example, it will be appreciated that other inflammatory GI conditions may be treated in a similar manner using a capsule 10 of embodiments and with similar advantages in improving the efficacy:toxicity balance.

1.-78. (canceled)

79. An ingestible capsule, comprising:
a housing, being a biocompatible indigestible housing including a therapeutic payload carrying compartment;
a power supply;
a release mechanism comprising a microcontroller and a release actuator;
and a sensing mechanism, the sensing mechanism being sensitive to an environment external to the housing;
the ingestible capsule being configured for passage through a gastrointestinal, GI, tract of a subject mammal, during which passage:
the sensing mechanism is configured to output an output signal varying according to the GI tract environment external to the housing;
wherein
either
A) the sensing mechanism comprises one or more from among a VOC gas sensor, a reflectometer formed by a transmission antenna of the ingestible capsule connected in series with a directional coupler configured to measure a reflected signal from the transmission antenna, and an accelerometer; and
the microcontroller is configured:
during an identification phase, on a rolling basis, to record a representation of the output signal for a most recent time period of duration t, and to process the recorded representation of the output signal for the most recent time period of duration t to identify presence of one or more ileocecal junction transition indicators, and
upon identification of the presence of the one or more ileocecal junction transition indicators, to determine the release timing and based on the determined release timing to cause the release actuator to release the therapeutic payload from the therapeutic payload carrying compartment;
and/or
B) the sensing mechanism comprises one or more from among an accelerometer, a TCD gas sensor, and a reflectometer formed by a transmission antenna of the ingestible capsule connected in series with a directional coupler configured to measure a reflected signal from the transmission antenna; and
the microcontroller is configured:
during an identification phase, on a rolling basis, to record a representation of the output signal for a most recent time period of duration t, and to process the recorded representation of the output signal for the most recent time period of duration t to identify presence of one or more gastric-duodenal transition indicators, and
upon identification of the presence of the one or more gastric-duodenal transition indicators, to determine the release timing and based on the determined release timing to cause the release actuator to release the therapeutic payload from the therapeutic payload carrying compartment.

80. The ingestible capsule according to claim 79, wherein
the sensing mechanism is a non-contact sensing mechanism housed in a portion of the ingestible capsule sealed from the environment external to the ingestible capsule by the housing, the non-contact sensing mechanism comprising at least one of an accelerometer and a reflectometer, the reflectometer comprising a transmission antenna connected in series with a directional coupler configured to measure a reflected signal from the transmission antenna, the output signal output by the sensing mechanism comprising accelerometer readings and/or reflectometer readings.

81. The ingestible capsule according to claim 80, wherein
the non-contact sensing mechanism comprises the reflectometer, and
the ingestible capsule further comprises a diode detector and the diode detector forms a part of the reflectometer, the diode detector being configured to receive the reflected signal from the antenna and to measure an amplitude of the reflected signal, the reflectometer readings in the output signal comprising amplitude measurements of the reflected signal.

82. The ingestible capsule of claim 81, wherein the ingestible capsule further comprises an antenna impedance control mechanism comprising a variable capacitor configured to vary impedance of the transmission antenna, and a controller, wherein the reflectometer and the antenna impedance control mechanism form a closed loop or feedback loop, and wherein the controller is configured to receive the measurements of the amplitude of the reflected signal from a diode detector and to execute a control algorithm to use the amplitude measurements to generate an antenna impedance control signal setting a capacitance of the variable capacitor to vary impedance of the antenna to reduce amplitude of the reflected signal, wherein the reflectometer readings in the output signal comprise readings of the antenna impedance control signal.

83. The ingestible capsule according to claim 80, wherein the output signal output by the sensing mechanism comprises accelerometer readings and reflectometer readings, and determining the release timing comprises identifying that an ileocecal junction transition indicator is present in readings from the reflectometer and the accelerometer, including:
processing the reflectometer readings and the accelerometer readings to identify the presence of a first ileocecal junction transition indicator in either one of the reflectometer readings and the accelerometer readings,
processing another one of the reflectometer readings and the accelerometer readings to identify a second ileocecal junction transition indicator within a predefined time window of a timing of the first ileocecal junction transition indicator, and
in response to identifying the first ileocecal junction transition indicator and the second ileocecal junction transition indicator within the predefined time window, determining the release timing, being either immediate or after a predefined delay.

84. The ingestible capsule according to claim 79, wherein
the or each ileocecal junction transition indicator and/or gastric-duodenal transition indicator is a characteristic or combination of characteristics of:
a reading, a series of readings, a pattern, a geometric feature, a statistical feature, and/or a mathematical feature, in a record of the output signal as a function of time;
the characteristic or combination of characteristics being predefined as being caused by transition of the ingestible capsule across the ileocecal junction or from a stomach into a duodenum.

85. The ingestible capsule according to claim 79, wherein
the therapeutic payload is one or more from among: a drug, a pharmaceutical formulation, a pre-biotic substance, a faecal transplant, and/or a pro-biotic substance.

86. The ingestible capsule according to claim 79, wherein
the ingestible capsule comprises a wireless transceiver, and the release mechanism comprises the wireless transceiver and a release actuator, and the antenna is configured, during an identification phase, to transmit a transmission signal representing the output signal to remote processing apparatus;
the transceiver being configured, upon receipt of a notification signal from the remote processing apparatus, to cause, immediately or at a predetermined delay, the therapeutic payload to be released from the therapeutic payload carrying compartment by the release actuator.

87. The ingestible capsule according to claim 79, wherein
the release mechanism comprises a microcontroller and a release actuator, and
the therapeutic payload carrying compartment comprises a section of the ingestible capsule housing and a sealed chamber, an elastic material membrane defining at least a portion of a wall of the sealed chamber, within which sealed chamber the therapeutic payload is sealed;
the release actuator comprising an elastic material membrane rupturing mechanism configured, at the determined release timing, to rupture the elastic material membrane thereby unsealing the sealed chamber, and allowing the therapeutic payload to exit the ingestible capsule via one or more apertures in the section of the ingestible capsule housing.

88. The ingestible capsule according to claim 87, wherein
the elastic material membrane rupturing mechanism comprises a power source and a heating element, the heating element arranged at least partially within, or against an interior surface of, the therapeutic payload carrying compartment, the at least a portion of the wall of the sealed chamber defined by the elastic material membrane being arranged to contact the heating element;
the power source being configured, at the determined release timing and under control of the microcontroller, to transfer energy to the heating element, to increase a temperature of the heating element and by which temperature increase to rupture the elastic material membrane, thereby unsealing the sealed chamber.

89. The ingestible capsule according to claim 88, wherein
the power source of the elastic material membrane rupturing mechanism is a supercapacitor configured to be trickle charged by the ingestible capsule power supply over a period of time beginning with an initiation event of the ingestible capsule, and to be caused to release the charge to the heating element at the determined release timing under the control of the microcontroller.

90. The ingestible capsule according to claim 87, wherein
the elastic material membrane rupturing mechanism comprises:
a power source and a LASER diode focussed on the elastic material membrane, wherein a microcontroller of the ingestible capsule is configured at the determined release timing to activate the LASER diode to rupture the elastic material membrane, thereby unsealing the sealed chamber; or
a pre-sprung mechanical needle, wherein a microcontroller of the ingestible capsule is configured, at the determined release timing, to release the pre-sprung mechanical needle causing the pre-sprung mechanical needle to spring into the elastic material membrane causing the elastic material membrane to rupture, thereby unsealing the sealed chamber.

91. The ingestible capsule according to claim 79, wherein
the release mechanism comprises a microcontroller and a release actuator, and
the therapeutic payload carrying compartment comprises a section of the ingestible capsule housing and a sealed chamber, an elastic material membrane defining at least a portion of a wall of the sealed chamber, within which sealed chamber the therapeutic payload is sealed;
the chamber is sealed by a releasable valve;
the release actuator comprises a releasable valve releasing mechanism configured, at the determined release timing, to release the releasable valve thereby unsealing the sealed chamber and allowing the therapeutic payload to exit the ingestible capsule via one or more apertures in the section of the ingestible capsule housing;
wherein
the releasable valve is a kinked hose, wherein the releasable valve releasing mechanism is a shape memory alloy wire or micro motor configured, under control of a microcontroller of the ingestible capsule, to unkink the hose thereby unsealing the chamber and allowing content of the chamber to exit the ingestible capsule via one or more apertures in the housing of the ingestible capsule.

92. The ingestible capsule according to claim 79, wherein
the release mechanism comprises a microcontroller and a release actuator, and
the therapeutic payload carrying compartment comprises a section of the ingestible capsule housing and a sealed chamber, an elastic material membrane defining at least a portion of a wall of the sealed chamber, within which sealed chamber a liquid diluent is sealed,
the therapeutic payload being a lyophilized drug or other therapeutic matter in powdered, dehydrated, or other solid form, and being contained within the therapeutic matter carrying compartment in a space external to the sealed chamber and at least partially defined by the elastic material membrane,
the section of the ingestible capsule housing comprising one or more apertures enabling fluid communication between the therapeutic payload carrying compartment and an exterior of the capsule, the one or more apertures being blocked by the elastic material membrane and unblocked following rupture of the elastic material membrane by an elastic material membrane rupturing mechanism at the determined release timing, the rupturing of the elastic material membrane allowing the liquid diluent to mix with the therapeutic payload within the therapeutic payload carrying compartment and to mix with fluids from the environment external to the capsule via the one or more apertures.

93. The ingestible capsule according to claim 79, wherein
the release timing is immediate upon identification of the ileocecal junction indicator; or
the release timing is a predetermined period following the identification of the ileocecal junction indicator; or
the release timing comprises a series of partial release timings at intervals, regular or irregular, wherein a fraction of the therapeutic payload is released at each of the partial release timings, and wherein an earliest partial release timing in the series of partial release timings is immediate upon identification of the ileocecal junction indicator or a predetermined period following identification of the ileocecal junction indicator.

94. A method comprising:
providing an ingestible capsule to a subject mammal for ingestion, wherein the ingestible capsule comprises a therapeutic payload;
processing an output signal from a sensing mechanism to determine a release timing of the therapeutic payload;
causing the therapeutic payload to be released into a GI tract of the subject mammal at the determined release timing;
wherein the ingestible capsule comprises:
a housing, being a biocompatible indigestible housing including a therapeutic payload carrying compartment;
a power supply;
a release mechanism comprising a microcontroller and a release actuator;
and a sensing mechanism, the sensing mechanism being sensitive to an environment external to the housing;
the ingestible capsule being configured for passage through a gastrointestinal, GI, tract of a subject mammal, during which passage:
the sensing mechanism is configured to output an output signal varying according to the GI tract environment external to the housing;
wherein
either
A) the sensing mechanism comprises one or more from among a VOC gas sensor, a reflectometer formed by a transmission antenna of the ingestible capsule connected in series with a directional coupler configured to measure a reflected signal from the transmission antenna, and an accelerometer; and
the microcontroller is configured:
during an identification phase, on a rolling basis, to record a representation of the output signal for a most recent time period of duration t, and to process the recorded representation of the output signal for the most recent time period of duration t to identify presence of one or more ileocecal junction transition indicators, and
upon identification of the presence of the one or more ileocecal junction transition indicators, to determine the release timing and based on the determined release timing to cause the release actuator to release the therapeutic payload from the therapeutic payload carrying compartment;
and/or
B) the sensing mechanism comprises one or more from among an accelerometer, a TCD gas sensor, and a reflectometer formed by a transmission antenna of the ingestible capsule connected in series with a directional coupler configured to measure a reflected signal from the transmission antenna; and
the microcontroller is configured:
during an identification phase, on a rolling basis, to record a representation of the output signal for a most recent time period of duration t, and to process the recorded representation of the output signal for the most recent time period of duration t to identify presence of one or more gastric-duodenal transition indicators, and
upon identification of the presence of the one or more gastric-duodenal transition indicators, to determine the release timing and based on the determined release timing to cause the release actuator to release the therapeutic payload from the therapeutic payload carrying compartment.

95. The method according to claim 94, wherein the release timing is a series of partial release timings, and a pharmaceutical formulation stored in the therapeutic payload is released into the GI tract by the release mechanism as a series of partial doses at the series of partial release timings.

96. A system comprising an ingestible capsule and a remote processing apparatus:
the ingestible capsule, comprising:
a housing, being a biocompatible indigestible housing including a therapeutic payload carrying compartment;
a power supply;
a release mechanism;
and a sensing mechanism, the sensing mechanism being sensitive to an environment external to the housing;
the ingestible capsule being configured for passage through a gastrointestinal, GI, tract of a subject mammal, during which passage:
the sensing mechanism is configured to output an output signal varying according to GI tract environment external to the housing; wherein
the release mechanism comprises a capsule transceiver and an actuator, and the capsule transceiver is configured, during an identification phase, to transmit a transmission signal representing the output signal to remote processing apparatus;
the capsule transceiver being configured, upon receipt of a notification signal from the remote processing apparatus, to cause, immediately or at a predetermined delay, the therapeutic payload to be released from the therapeutic payload carrying compartment by the release actuator; and
the remote processing apparatus comprising a remote processing apparatus transceiver configured to communicate with the capsule transceiver including to receive the transmission signal representing the output signal, and a processor configured to process the output signal to identify in real time either:
A) one or more ileocecal junction transition indicators wherein the sensing mechanism comprises one or more sensors from among a VOC gas sensor, a reflectometer formed by a transmission antenna of the ingestible capsule connected in series with a directional coupler configured to measure a reflected signal from the transmission antenna, and an accelerometer; and/or
B) one or more gastric duodenal transition indicators in the output signal, wherein the sensing mechanism comprises one or more from among an accelerometer, a TCD gas sensor, and a reflectometer formed by a transmission antenna of the ingestible capsule connected in series with a directional coupler configured to measure a reflected signal from the transmission antenna;
and the processor is further configured:
to cause the remote processing apparatus transceiver to transmit the notification signal to the capsule transceiver according to the identification of the one or more ileocecal junction transition indicators and/or the one or more gastric duodenal transition indicators.

97. The system according to claim 96, wherein
the sensing mechanism is a non-contact sensing mechanism housed in a portion of the ingestible capsule sealed from the environment external to the ingestible capsule by the housing, the non-contact sensing mechanism comprising at least one of an accelerometer and a reflectometer, the reflectometer comprising a transmission antenna connected in series with a directional coupler configured to measure a reflected signal from the transmission antenna, the output signal output by the sensing mechanism comprising accelerometer readings and/or reflectometer readings.

98. The system according to claim 97,
wherein the output signal output by the sensing mechanism comprises accelerometer readings and reflectometer readings, and determining the release timing comprises identifying that an ileocecal junction transition indicator is present in readings from the reflectometer and the accelerometer, including:
processing the reflectometer readings and the accelerometer readings to identify a presence of a first ileocecal junction transition indicator in either one of the reflectometer readings and the accelerometer readings,
processing another one of the reflectometer readings and the accelerometer readings to identify a second ileocecal junction transition indicator within a predefined time window of a timing of the first ileocecal junction transition indicator, and
in response to identifying the first ileocecal junction transition indicator and the second ileocecal junction transition indicator within the predefined time window, determining the release timing, being either immediate or after a predefined delay.
